SEQUENCE LISTING (20 sequences)

SEQ ID NO: 1 - DNA, 945 bp, Homo sapiens
atggggctcctggactcggagccgggtagtgtcctaaacgtagtgtccacggcactcaacgacacggtagagttctaccgctggacctggtccatcgcagataagcgtgtggaaaattggcctctgatgcagtctccttggcctacactaagtataagcactctttatctcctgtttgtgtggctgggtccaaaatggatgaaggaccgagaaccttttcagatgcgtctagtgctcattatctataattttgggatggttttgcttaacctctttatcttcagagagttattcatgggatcatataatgcgggatatagctatatttgccagagtgtggattattctaataatgttcatgaagtcaggatagctgctgctctgtggtggtactttgtatctaaaggagttgagtatttggacacagtgttttttattctgagaaagaaaaacaaccaagtttctttccttcatgtgtatcatcactgtacgatgtttaccttgtggtggattggaattaagtgggttgcaggaggacaagcattttttggagcccagttgaattcctttatccatgtgattatgtactcatactatgggttaactgcatttggcccatggattcagaaatatctttggtggaaacgatacctgactatgttgcaactgattcaattccatgtgaccattgggcacacggcactgtctctttacactgactgccccttccccaaatggatgcactgggctctaattgcctatgcaatcagcttcatatttctctttcttaacttctacattcggacatacaaagagcctaagaaaccaaaagctggaaaaacagccatgaatggtatttcagcaaatggtgtgagcaaatcagaaaaacaactcatgatagaaaatggaaaaaagcagaaaaatggaaaagcaaaaggagattaa

SEQ ID NO: 2 - protein, 314 aa, Homo sapiens
MGLLDSEPGSVLNVVSTALNDTVEFYRWTWSIADKRVENWPLMQSPWPTLSISTLYLLFVWLGPKWMKDREPFQMRLVLIIYNFGMVLLNLFIFRELFMGSYNAGYSYICQSVDYSNNVHEVRIAAALWWYFVSKGVEYLDTVFFILRKKNNQVSFLHVYHHCTMFTLWWIGIKWVAGGQAFFGAQLNSFIHVIMYSYYGLTAFGPWIQKYLWWKRYLTMLQLIQFHVTIGHTALSLYTDCPFPKWMHWALIAYAISFIFLFLNFYIRTYKEPKKPKAGKTAMNGISANGVSKSEKQLMIENGKKQKNGKAKGD

SEQ ID NO: 3 - DNA, 939 bp, Mus Musculus
atggggctgctggactcagagcccggcagcgtcctgaacgcgatgtccacggcattcaacgacaccgtggagttctatcgctggacctggaccattgcagataaacgtgtagcagactggccgctgatgcagtctccatggccaacgataagcataagcacgctctatctcctgttcgtgtggctgggtccaaagtggatgaaagaccgcgagccgttccaaatgcgcttagtactcataatctataattttggcatggttttgcttaaccttttcatcttcagagagctattcatgggatcatacaacgcaggatacagctatatttgccagtcagtggattattctaatgatgttaatgaagtcaggatagcggcggccctgtggtggtattttgtatcgaaaggcgttgagtatttggacacagtgttttttatcctgaggaagaaaaacaaccaagtctccttccttcacgtgtaccaccactgcaccatgttcactctgtggtggattggaatcaagtgggtggctggaggccaagcgtttttcggggcccagatgaactctttcatccacgtgatcatgtactcctactatgggctgactgcgttcggcccctggatccagaaatatctttggtggaagcgatacctgaccatgctgcagctggtccagttccacgtgaccatcggacacacagcactgtctctctacaccgactgccccttccccaagtggatgcactgggctctgatcgcctacgccatcagcttcatcttcctcttcctcaacttctacactcggacatacaatgagccgaagcagtcaaaaaccggaaagacggccacgaatggtatctcatcgaacggcgtgaataaatcagagaaagcgttagaaaacgggaaaccccagaaaaacgggaagccaaaaggagagtaa

SEQ ID NO: 4 - protein, 312 aa, Mus Musculus
MGLLDSEPGSVLNAMSTAFNDTVEFYRWTWTIADKRVADWPLMQSPWPTISISTLYLLFVWLGPKWMKDREPFQMRLVLIIYNFGMVLLNLFIFRELFMGSYNAGYSYICQSVDYSNDVNEVRIAAALWWYFVSKGVEYLDTVFFILRKKNNQVSFLHVYHHCTMFTLWWIGIKWVAGGQAFFGAQMNSFIHVIMYSYYGLTAFGPWIQKYLWWKRYLTMLQLVQFHVTIGHTALSLYTDCPFPKWMHWALIAYAISFIFLFLNFYTRTYNEPKQSKTGKTATNGISSNGVNKSEKALENGKPQKNGKPKGE

SEQ ID NO: 5 - DNA, 945 bp, Macaca
atggggctcctggactcggagccgggtagtgtcctaaacgtagtgtccacggcactcaacgacacggtggagttctaccgctggacctggtccatcgcagataagcgtgtggagaattggcctctgatgcagtctccttggcctacactaagtataagcactctttatctcctgtttgtgtggctgggtccaaagtggatgaaggaccgagaaccttttcagatgcgtctagtgctcattatctataattttgggatggttttgcttaacttttttatcttcagagagttattcatgggatcatataatgcgggatatagctatatttgccagagtgtggattattctaataatgttaatgaagtcaggatagctgctgctctgtggtggtactttgtttctaaaggagttgagtatttggacacagtgttttttattctgagaaagaaaaacaaccaggtttctttccttcatgtgtatcatcactgtacgatgtttaccttgtggtggattggaattaagtgggttgcaggaggacaagcattttttggagcccagatgaattcctttatccatgtgattatgtactcatactatgggttagctgcatttggcccatggattcagaaatatctttggtggaaacgatacctgactatgttgcaactggttcaattccatgtgaccattgggcacacagcactgtctctttacactgactgccccttccccaaatggatgcactgggctctaattgcctacgcaatcagcttcatatttctctttcttaacttctacattcggacatacaaagagcctaagaaaccaaaaactggaaaaacagccatgaatggtatttcagcaaatggtgtgagcaaatcagaaaaacaactcgtgatagaaaacggaaaaaagcagaaaaatggaaaagcaaaaggagattaa

SEQ ID NO: 6 - protein, 314 aa, Macaca
MGLLDSEPGSVLNVVSTALNDTVEFYRWTWSIADKRVENWPLMQSPWPTLSISTLYLLFVWLGPKWMKDREPFQMRLVLIIYNFGMVLLNFFIFRELFMGSYNAGYSYICQSVDYSNNVNEVRIAAALWWYFVSKGVEYLDTVFFILRKKNNQVSFLHVYHHCTMFTLWWIGIKWVAGGQAFFGAQMNSFIHVIMYSYYGLAAFGPWIQKYLWWKRYLTMLQLVQFHVTIGHTALSLYTDCPFPKWMHWALIAYAISFIFLFLNFYIRTYKEPKKPKTGKTAMNGISANGVSKSEKQLVIENGKKQKNGKAKGD

SEQ ID NO: 7 - DNA, 945 bp, Bos taurus
atggggctcctggactcggagccgggcagcgtcctgaacgcggtgtccacggcgctgaacgacaccgtggagttctaccgctggaccttgtccatcacagataaacgcgtcgagaactggcctctgatgcagtctccactgcctacactttgcataagcaccctgtacctcctcttcgtgtggctgggtccaaaatggatgaaggaccgagaacctttccagatgcgtttagtgctcattctctataatttcgggatggttttgcttaacctttttatcttcagagagttactcatgggatcatataatgcaggatatagctatatttgccagactgtggattattctgataacgttcatgaagtcaggatagctgctgctctgtggtggtactttatatctaaaggaattgagtatttggacacggtgttttttatcctgaggaagaaaaacaaccaagtctcttttcttcacgtgtatcatcactgtaccatgtttacgttgtggtggattggaattaagtgggttgcaggaggacaggcattttttggagcccagataaattccttcatccatgtgattatgtattcatactatgggttagctgccttcggcccgtggattcagaaatatctctggtggaaacgatacctgaccatgttgcagctggttcagttccacgtgaccattgggcacacagcactgtccctttacaccgactgccccttccccaagtggatgcactgggctctcattgtctatgcagtcagcttcatattcctcttccttaacttctatgtgcggacgtacaaagagcctaagaaagcaaaacccggaaaaacagcaacgaatggtatttcagctaatggtgtgaacaaatccgaaaaccacctagtggtagaaaatggcaagaagcagaagaatggaaaagcaaaaggagagtaa

SEQ ID NO: 8 - protein, 314 aa, Bos taurus
MGLLDSEPGSVLNAVSTALNDTVEFYRWTLSITDKRVENWPLMQSPLPTLCISTLYLLFVWLGPKWMKDREPFQMRLVLILYNFGMVLLNLFIFRELLMGSYNAGYSYICQTVDYSDNVHEVRIAAALWWYFISKGIEYLDTVFFILRKKNNQVSFLHVYHHCTMFTLWWIGIKWVAGGQAFFGAQINSFIHVIMYSYYGLAAFGPWIQKYLWWKRYLTMLQLVQFHVTIGHTALSLYTDCPFPKWMHWALIVYAVSFIFLFLNFYVRTYKEPKKAKPGKTATNGISANGVNKSENHLVVENGKKQKNGKAKGE

SEQ ID NO: 9 - DNA, 945 bp, Equus caballus
atggggctcctggacgcggagccaggcagtgtcctgaacgtggtgtccacggcgctcaacgacaccgtggagttgtaccgctggacctgttctttcgcagataagcgtgtggaaaactggcctctgatgcagtccccatggactacgcttggtatcagcactctttatctcctgttcgtgtggctgggtccaaaatggatgaaggaccgagagccttttcagatgcgcttagtgctcattatctataattttggcatggttttccttaacctttttatcttcagagagttattcatgggatcatataatgcaggatatagctatatttgccagactgtggattattctgataatgttcacgaagtcaggatagctgctgctctgtggtggtactttgtgtctaaaggagtggagtatttagacacggtgttttttatcttgaggaagaaaaacaatcaagtctctttccttcatgtatatcatcactgtacaatgtttacgttgtggtggattggaattaagtgggtcgcagggggacaagcttttttcggagcccagatgaattccctcatccatgtgattatgtactcctactacggcttaagtgcgtttggcccatggattcagaagtatctttggtggaaacgatacctgactatgttgcagctgatccagttccatgtgaccattggccacacagcactgtctctgtacacggattgccctttccccaaatggatgcactgggctctcattgtctattcaatcagcttcatacttctctttcttaacttctatgttcggacatacaacatgcctaagaaatcccaaactggaaaaccagctgtgaatggtatttcagcaaatggtgtgagcaaatcagaaaaacagctagtgatggaaaatggaaaaaagcagaaaactggaaaagcaaaaggagagtaa

SEQ ID NO: 10 - protein, 314 aa, Equus caballus
MGLLDAEPGSVLNVVSTALNDTVELYRWTCSFADKRVENWPLMQSPWTTLGISTLYLLFVWLGPKWMKDREPFQMRLVLIIYNFGMVFLNLFIFRELFMGSYNAGYSYICQTVDYSDNVHEVRIAAALWWYFVSKGVEYLDTVFFILRKKNNQVSFLHVYHHCTMFTLWWIGIKWVAGGQAFFGAQMNSLIHVIMYSYYGLSAFGPWIQKYLWWKRYLTMLQLIQFHVTIGHTALSLYTDCPFPKWMHWALIVYSISFILLFLNFYVRTYNMPKKSQTGKPAVNGISANGVSKSEKQLVMENGKKQKTGKAKGE

SEQ ID NO: 11 - DNA, 945 bp, Gallus gallus
atgggtgccgccgccgccgagcccccccgcgccgcgggcctggtgtccagcgtgctgaacgacaccctcgagttctaccgctggacctggagcatccgagacaaacgtgtcgatgattggcccttgatgcagtctccatttccaacactgactataagcactatttatctcctcactgtttggctgggccccaaatggatgaagacaagagagcccttccagttacgtttcctgttggttgtttacaactttggaatggttctactcaacttcttcattttcaaagagctgtttttatcatcaagagctcgagggtacagctatgtctgccagactgtggattattcagataatgtttatgaagttaggatagctgctgctttatggtggtattatgtctccaaaggaattgaatacctagatacagtcttcttcatcctgaggaagaagttcaaccaaattagttttcttcatgtctatcaccatttcaccatgttcactttgtggtggattggtattaagtgggttgcaggtggacaagctttttttggagctcagatgaatgcatttattcatgttattatgtacatgtattacggattagctgcctgtggccctaaagttcagaagtatctgtggtggaaacgatacttgactatattgcaactggtgcagttccatgtgactattggccacacagccttgtctatttatatcgattgtcctttccctaaatggatgcactggggtgtaattttctatgctatcaccttcattttcttgtttggtaacttctactatcggacatataagctgcccaaggaacctgtaaagaatggcaaaatagcaaatggtgctgttgcaaacggagtaagcaaaccagaaaataatccagtggtggaaaatggaaaaaagcagaaaaagggaaaagcaaaaggagagtaa

SEQ ID NO: 12 - protein, 313 aa, Gallus gallus
MGAAAAEPPRAAGLVSSVLNDTLEFYRWTWSIRDKRVDDWPLMQSPFPTLTISTIYLLTVWLGPKWMKTREPFQLRFLLVVYNFGMVLLNFFIFKELFLSSRRGYSYVCQTVDYSDNVYEVRIAAALWWYYVSKGIEYLDTVFFILRKKFNQISFLHVYHHFTMFTLWWIGIKWVAGGQAFFGAQMNAFIHVIMYMYYGLAACGPKVQKYLWWKRYLTILQLVQFHVTIGHTALSIYIDCPFPKWMHWGVIFYAITFIFLFGNFYYRTYKLPKEPVKNGKIANGAVANGVSKPENNPVVENGKKQKKGKAKGE

SEQ ID NO: 13 - DNA, 912 bp, Danio rerio
atggagacggtcgttcacctgatgaatgactctgtagagttttacaaatggagccttaccatagcagacaagcgtgtggagaaatggccgatgatgtcatctcctctgcccactctggggatcagtgttttgtacctgctcttcctttgggccggccctctttacatgcagaaccgcgagcctttccagctcaggaaaacactcattgtgtacaacttcagcatggtgctgcttaacttctacatctgcaaagagctgctcctgggctccagagcagccggatacagctacctctgccagcctgtcaactactccaatgatgttaatgaagtcaggatagcatctgctctgtggtggtattacatctccaagggagtggagtttctggacacggtcttcttcatcatgaggaagaagtttaatcaggtcagcttcctgcacgtctatcaccactgcacaatgttcatcctgtggtggatcggcatcaagtgggttcctggtggacagtctttctttggcgcaacgattaactcaggcattcatgtgctgatgtacggctactacggcctggcagcgtttggcccgaagatccagaagtacctgtggtggaagaaatacctcactattattcagatgatccagttccacgtcaccattggccatgctgctcactctctctacacgggctgtccattcccagcatggatgcagtgggctttgattggctatgccgttacattcatcatcctgttcgccaatttttactaccagacctaccgtcgccagccacgtctcaagacagccaaatccgcagttaacggcgtctccatgtcaaccaacggcaccagcaagacagccgaggtcacggaaaatggaaagaaacagaagaaaggaaaaggaaagcacgattaa

SEQ ID NO: 14 - protein, 303 aa, Danio rerio
METVVHLMNDSVEFYKWSLTIADKRVEKWPMMSSPLPTLGISVLYLLFLWAGPLYMQNREPFQLRKTLIVYNFSMVLLNFYICKELLLGSRAAGYSYLCQPVNYSNDVNEVRIASALWWYYISKGVEFLDTVFFIMRKKFNQVSFLHVYHHCTMFILWWIGIKWVPGGQSFFGATINSGIHVLMYGYYGLAAFGPKIQKYLWWKKYLTIIQMIQFHVTIGHAAHSLYTGCPFPAWMQWALIGYAVTFIILFANFYYQTYRRQPRLKTAKSAVNGVSMSTNGTSKTAEVTENGKKQKKGKGKHD

SEQ ID NO: 15 - DNA, 660 bp, Xenopus
atgaagtcagggtggcctcagctctctggtggtactatgtttcaaaaggggtggaatattttgacacagtatttttcatacttagaaaaaaaattcaaccaaattagttttctgcatgtatatcaccactgcacaatgttcacactctggtggatcggaatcaaatgggttgccggaggacaatcattttttggagcgcacatgaatgcactgatccatgttgtgatgtacttatattatgggttggctgcctgtggaccacatctacagaagtatttatggtggaaacgatatctgacaatattgcagctggttcaattccatgtgacaatcggacatacagctttatcactgtacattgattgtccatttcccaagtggatgcattgggctctgattgtgtatgctatcacttttattattcttttcgttaacttctactaccggacatacaatgctcccaaagcaccagctaaaagtgggaaatcactcataaatggaaagacctctgttaatggcaagtcctctgtgaatggaaaatgccaaattaatggaaaattaatgaatggagcagttaatggagctgtaagcaagcaagataataaagttggacaagaaaatgggcgaaaaaggagaaaaggaagagccaagcgagaataa

SEQ ID NO: 16 - protein, 219 aa, Xenopus
MKSGWPQLSGGTMFQKGWNILTQYFSYLEKKFNQISFLHVYHHCTMFTLWWIGIKWVAGGQSFFGAHMNALIHVVMYLYYGLAACGPHLQKYLWWKRYLTILQLVQFHVTIGHTALSLYIDCPFPKWMHWALIVYAITFIILFVNFYYRTYNAPKAPAKSGKSLINGKTSVNGKSSVNGKCQINGKLMNGAVNGAVSKQDNKVGQENGRKRRKGRAKRE

SEQ ID NO: 17 - DNA, 1038 bp, Yeast
atgaacactaccacatctactgttatagcagcagttgccgaccagttccagtctttgaactcttcttcttcatgtttcttgaaggttcatgttccttccattgagaacccattcggtattgaattatggccaattttctccaaagtgtttgaatactttagtggctatccagctgagcaattcgagtttattcacaataagactttcttggctaacgggtatcatgctgttagtattattatcgtttattacattattatctttggtggccaagctatcttacgcgccttgaacgcctctccattaaagtttaaattgcttttcgagatacacaacttgtttttgacttctatttctctagttttatggttgctgatgttagaacagttggttcctatggtttatcacaacggtctattctggtctatctgctctaaggaagccttcgcaccaaaattagttactctttactatttgaactatttgaccaaattcgtagaattgattgacactgtgtttttagttttgagaagaaagaagttattgtttttgcacacttaccatcacggtgccaccgctttgttgtgctacactcaattaattggtcgtacttctgttgaatgggtagttatcctactaaacttgggtgttcacgttatcatgtactggtactacttcttgagttcatgtggtattagagtttggtggaagcaatgggtcactagattccaaattattcaatttttgattgacttggtatttgtttactttgctacctatacattctatgctcacaaatacttggacggtattttaccaaacaagggtacttgttatggtactcaggctgctgctgcttatgggtatttgattctaacatcttatttgcttttgtttatttccttctacatccaatcttacaagaaaggtggtaaaaagacagtcaagaaggaatctgaagtttccggctccgttgcatccggttcttctactggtgtcaagacctctaacaccaaggtctcttccaggaaagcttaa

SEQ ID NO: 18 - protein, 345 aa, Yeast [sequence omitted]

SEQ ID NO: 19 - protein, 5 aa, Artificial
  Completely synthesized.
KXKXX

SEQ ID NO: 20 - protein, 5 aa, Artificial
  Completely synthesized.
HXXHH

(19) United States
(12) Patent Application Publication
(10) Pub. No.: US 2014/0100280 A1
(43) Pub. Date: Apr. 10, 2014

(54) TREATMENT METHODS USING VERY LONG CHAIN POLYUNSATURATED FATTY ACIDS

(71) Applicant: THE BOARD REGENTS OF THE UNIVERSITY OF OKLAHOMA, NORMAN, OK (US)

(72) Inventors: Robert E. Anderson, Edmond, OK (US); Martin-Paul Agbaga, Oklahoma City, OK (US); Richard S. Brush, Midwest City, OK (US)

(73) Assignee: THE BOARD REGENTS OF THE UNIVERSITY OF OKLAHOMA, NORMAN, OK (US)

(21) Appl. No.: 14/074,859

(22) Filed: Nov. 8, 2013

Related U.S. Application Data
(63) Continuation-in-part of application No. 13/231,649, filed on Sep. 13, 2011, now abandoned.
Application No. 13/231,649 is a continuation-in-part of application No. 12/361,163, filed on Jan. 28, 2009, now Pat. No. 8,021,874.
(60) Provisional application No. 61/062,579, filed on Jan. 28, 2008.

(51) Int. Cl. A61K 31/20

(52) U.S. Cl. CPC A61K 31/20
USPC 514/560

(57) ABSTRACT

Compositions that include Very Long Chain Polyunsaturated Fatty Acids (VLC-PUFAs) are disclosed, along with methods of production and use thereof.

### CROSS-REFERENCE TO RELATED APPLICATIONS/INCORPORATION BY REFERENCE STATEMENT

The present application is a continuation-in-part of U.S. Ser. No. 13/231,649, filed Sep. 13, 2011, now abandoned; which is a continuation-in-part of U.S. Ser. No. 12/361,163, filed Jan. 28, 2009, now U.S. Pat. No. 8,021,874, issued Sep. 20, 2011; which claims benefit under 35 U.S.C. §119(e) of U.S. Provisional Application Ser. No. 61/062,579, filed Jan. 28, 2008. The entire contents of each of the above-referenced patents and patent applications are hereby expressly incorporated herein by reference.

### STATEMENT REGARDING FEDERALLY SPONSORED RESEARCH OR DEVELOPMENT

This invention was made with government support under Contract Numbers EY004149, EY000871, EY012190, and RR017703 awarded by the National Institutes of Health (NIH). The government has certain rights in the invention.

### BACKGROUND

Very long chain polyunsaturated fatty acids (VLC-PUFAs), with their structurally unusual long hydrocarbon chains, are essential lipids that play important roles in certain biological systems that cannot be fulfilled by the more common shorter chain C16-C18 fatty acids²⁵. Because of their very long chain structure, some VLC-PUFAs are able to span and reside within both leaflets of the lipid bilayer, thereby giving stability to highly curved cellular membranes such as those which surround nuclear pore complexes²⁵. In photoreceptors, for example, the VLC-PUFAs are known to be associated with rhodopsin and play a role in regulation of phototransduction cascades^{18,21}. Absence of these VLC-PUFAs appears to contribute to macular degeneration in autosomal dominant Stargardt macular dystrophy (STGD3, MIM600110)¹⁸, for example.

Three independent mutations in exon six of the Elongation of Very Long chain fatty acids-4 (ELOVL4) gene are associated with STGD3^{1,2}. These mutations have been shown to cause truncation and subsequent mislocalization and aggregation of the normal ELOVL4 protein product³⁻⁵. Based on sequence homology with a group of functional yeast genes and other mammalian ELOVLs, the ELOVL4 protein was predicted to be involved in elongation of very long chain fatty acids (VLCFAs)^{1,4,6}. For example, the microsomal ELO1 (Ssc1) is responsible for elongation of 14:0 to 16:0^{7,8}. ELO2 (Ssc2), ELO3 (Cig30), and ELOVL5 have been shown to be involved in elongation of saturated, monounsaturated, or polyunsaturated fatty acids from 18 to 26 carbons⁸⁻¹⁰. However, whether the ELOVL4 protein is involved in fatty acid elongation, and the specific step it may catalyze, has remained unknown^{9,11}. Based on its high level of expression in retinal photoreceptor cells, and to lesser extents in brain, testis and skin, it was first hypothesized that ELOVL4 may be involved in the biosynthetic pathway of docosahexaenoic acid (22:6n3, DHA), the most abundant polyunsaturated fatty acid in the retina and the brain¹. However, recent experiments carried out in our laboratory (unpublished data) and results obtained from ELOVL4 mutant mice¹²⁻¹⁴ do not support this hypothesis.

Current reports establish ELOVL4 as an essential protein for growth and development as homozygous ELOVL4 knock-out or knock-in of ELOVL4 mutant genes results in neonatal lethality in mice¹²⁻¹⁵. The heterozygote knock-in mouse, in which the mutant human ELOVL4 gene has replaced one wild type copy, develops progressive photoreceptor degeneration similar to human STGD3, demonstrating its association with the function of photoreceptors¹⁶. Homozygous neonates exhibit scaly wrinkled skin due to severely compromised epidermal permeability barrier and die within hours of birth^{12-15,17}. Lipid analysis of the epidermis from the homozygote ELOVL4 knock-out mice indicated a global reduction in very long chain saturated and hydroxy fatty acids longer than 26:0 in omega hydroxyl ceramides/glucosylceramides and free fatty acid fractions¹²⁻¹⁵. Also, mice with one normal gene replaced with a gene containing the STGD3 disease mutation had lower amounts of C32-C36 acyl phosphatidylcholines in their retinas¹⁸. Based on these findings it was proposed that the ELOVL4 protein functions in VLC-FA and/or VLC-PUFA biosynthesis^{14,15,18}. However, none of the studies published prior hereto have provided direct evidence that ELOVL4 is an elongase. Rather, previous work has only shown that absence or reduced expression of ELOVL4 leads to a reduction in the levels of certain fatty acids. This result could have several explanations not related to elongase activity. For example, ELOVL4 protein could provide some co-factor necessary for VLC-FA/PUFA synthesis or ELOVL4 protein could support activity of a cellular organelle necessary for VLC-FA/PUFA synthesis.

Moreover, research into the VLC-PUFA class of fatty acids has been restricted due to the unavailability of these fatty acids, and their use in commercial formulations and compositions has not been feasible due to the lack of known processes for synthesizing them. It is to the development of such synthetic methods and the compositions and uses of VLC-PUFAs produced therefrom, that certain embodiments of the presently disclosed and claimed inventive concept(s) are directed.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1: Adenovirus efficiently transfers ELOVL4 protein to rat cardiac myocytes and ARPE-19 cells. (a) Expression values are determined by qRT-PCR and presented over the expression of house-keeping gene Rpl 19. B, brain; R, retina; S, skin; T, testis; L, liver; H, heart; C, cardiomyocytes; C-E, cardiomyocytes transduced with Elovl4; A19, ARPE19; R-C, RPE-Choroid. (b) Immunofluorescent images showing efficient adenoviral gene transfer in cardiac myocytes top row and ARPE-19 cells (middle row) infected with Ad5-mELOVL4 and cardiac myocytes infected with Ad5-GFP (bottom row); these are stained for mouse ELOVL4 (top and middle rows) and GFP protein (bottom row) 24 h post infection. The column on the left represents nuclei stain for DAPI (4′,6-diamidino-2-phenylindole). The middle column represents ELOVL4 protein stains. The column on the right represents merged images from left and middle columns. Arrows represent some non-infected cells that do not express ELOVL4 or GFP proteins. (c) Western blots showing ELOVL4 and GFP expression in transduced and non-transduced cells. Lanes: 1. myocytes non-transfected; 2. myocytes+Ad5-mELOVL4 24 h treatment; 3. myocytes+Ad5-mELOVL4 72 h treatment; 4. myocytes+Ad5-GFP 24 h treatment; 5. myocytes+Ad5-GFP 72 h treatment.

FIG. 2: Synthesis of 28:0 and 30:0 fatty acids from 24:0 fatty acid supplementation is enhanced in myocytes expressing mouse Elovl4 transgene. Two controls (GFP expressing and non-transduced myocytes) and mouse ELOVL4 expressing myocytes were treated without (a) or with (b) 24:0 for 72 h. While there were no significant differences between the levels of 28:0 and 30:0 between the control groups, there were significant differences in levels of 28:0 and 30:0 in mouse ELOVL4 expressing myocytes compared to the two controls. **p<0.01, ***p<0.001, (n=3).

FIG. 3: Elongation of very long chain fatty acids is enhanced in ARPE-19 cells expressing mouse ELOVL4. ARPE-19 cells non-transduced, Ad5-GFP or Ad5-mELOVL4 transduced were treated without 24:0 fatty acids (a) or with 24:0 fatty acids (b) for 48 h. There were no significant differences in levels of 28:0 and 30:0 fatty acids in the normal ARPE-19 or GFP expressing cells with or without 24:0 supplementation. ELOVL4 expression in the cells resulted in 7.5 fold increase in 28:0 with or without 24:0 supplementation. 30:0 fatty acid was increased 20 fold in ELOVL4 expressing cells and upon treatment with 24:0 (b) there was a 50 fold increase in the amount of 30:0 made compared to the two controls. ***p<0.001, (n=3).

FIG. 4: GC/MS chromatograms illustrating VLC-PUFAs synthesized from 20:5n3 and 22:5n3 fatty acid supplementation in ELOVL4 transduced cells. (a) Cardiomyocytes transduced with mouse ELOVL4 (“Myo ELO1”), GFP transduced cells (“Myo GFP2”) and non-transduced cells (“Myo 3”) cultured without precursors for 72 h. Presence of ELOVL4 in absence of precursor resulted in elongation of endogenous precursor to generate VLC-PUFAs. Abundances were compared by normalizing chromatograms using the peak height of 20:1 (a fatty acid confirmed to be unaffected by the experimental process). (b) Incubation of the cells with 20:5n3 (EPA) resulted in significant biosynthesis of C28-C38 n-3 VLC-PUFAs in ELOVL4 (“ELO+EPA 30A”) transduced cells but not in GFP (“GFP+EPA 30A”) and non-transduced cells (“EPA 30A”). (c) Addition of 22:5n3 (DPA) also resulted in the synthesis of n-3 VLC-PUFAs, showing no observable differences from those synthesized in the presence of 20:5n3.

### DETAILED DESCRIPTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation, and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the method described herein without departing from the concept, spirit and scope of the presently disclosed and claimed inventive concept(s). All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The singular forms “a,” “an,” and “the” include plural referents unless the context clearly indicates otherwise. Thus, for example, reference to “a compound” may refer to 1 or more, 2 or more, 3 or more, 4 or more or greater numbers of compounds. The term “plurality” refers to “two or more.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by ±20% or ±10%, or ±5%, or ±1%, or ±0.1% from the specified value, as such variations are appropriate to perform the disclosed methods and as understood by persons having ordinary skill in the art. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

Turning now to the presently disclosed and claimed inventive concept(s), certain embodiments thereof are related to processes for the production of Very Long Chain Polyunsaturated Fatty Acids (VLC-PUFAs). Certain embodiments of the presently disclosed and claimed inventive concept(s) relate to compositions (e.g., nutritional supplements, food products, and pharmaceutic compositions) containing such VLC-PUFAs. In at least one embodiment, the presently disclosed and claimed inventive concept(s) is directed to methods for biosynthesis and production of the VLC-PUFAs described herein (particularly C28-C40 PUFAs, also referred to herein as supraenes or supraenoics); the methods may include expression of the full or partial sequence(s) of ELOVL4 DNA/mRNA nucleic acids or ELOVL4 protein sequences encoded thereby in a production host cell from any species (prokaryotic or eukaryotic). The method may be used in the biosynthesis, production, purification, and/or utilization of VLC-PUFAs, and in particular, by the elongation of C18-C26 saturated fatty acids and PUFAs. In certain embodiments, the presently disclosed and claimed inventive concept(s) may comprise a dietary supplement, a food product, a pharmaceutical formulation, a humanized animal milk, an infant formula, and/or a cosmetic item that comprises any of the compositions described or otherwise contemplated herein. A pharmaceutical formulation can include, but is not limited to, a drug for treatment of a neurodegenerative disease, a retinal disorder, age related maculopathy, a fertility disorder particularly regarding sperm or testes, and/or a skin disorder.

The processes of some embodiments of the presently disclosed and claimed inventive concept(s) include, but are not limited to, the use of any eukaryotic, prokaryotic, or viral promoter(s) or enhancer(s) element known by persons of ordinary skill in the art to drive the production of expression of the ELOVL4 genes or proteins in host production cells (such as, but not limited to, algae, bacteria, yeast, plant, insect, worm, and mammalian cells). The cells may be transduced, infected, transfected, or otherwise transformed with the Elovl4 gene (or other similar sequences defined herein) and have genes for the expression of enzymes for elongating C18-C26 fatty acids, and the cells may be grown in the presence of specific n3 and/or n6 fatty acid precursors (including, but not limited to, 18:3n3, 20:5n3, 22:5n3, 22:6n3, 18:2n6, 20:4n6, 22:4n6, 22:5n6, 24:5n3, 24:6n3, 24:4n6, 26:5n3, 26:6n3, 26:7n3, 26:5n6, or 26:6n6).

Very Long Chain Polyunsaturated Fatty Acids (VLC-PUFAs) having carbon chains of C28 to C40, while found in a number of species and organs therein (e.g., testes, retinas, brain, and sperm), are present in extremely small quantities. Prior to the present work, there have been no methods available for synthetically producing these VLC-PUFAs (much less in commercial quantities). In order to obtain even minute μg quantities of these VLC-PUFAs, they must be extracted from natural sources such as bovine retinas. As a result, research into C28-C40 VLC-PUFAs has been limited, and means for commercial production thereof have been non-existent. For example, ten bovine retinas typically yield roughly 500 μg of mixed VLC-PUFAs. The process involves discontinuous sucrose centrifugation to separate the photoreceptor outer segments (where the VLC-PUFAs are concentrated) from the rest of the retina, total lipid extraction to isolate lipids from the outer segments, derivatization to hydrolyze the lipids and generate fatty acid methyl esters (FAME), silver nitrate TLC to separate the VLC-PUFAs from undesired fatty acids, and reverse-phase TLC to further purify the VLC-PUFAs. This is a very time-consuming and labor intensive process to obtain only a very small quantity of VLC-PUFAs. Having a means of generating and obtaining large amounts of these VLC-PUFAs as contemplated and described herein is not only novel but is greatly desired.

In the present work, it has been established that ELOVL4 protein encoded by the Elovl4 gene catalyzes or co-catalyzes production of VLC-PUFAs by elongation of shorter chain (C18-C26) fatty acids. Based on this finding, recombinant processes have been invented for synthetically producing C28-C40 VLC-PUFAs in significant quantity for use in purified compositions as well as in commercial compositions for use as nutritional formulas and/or supplements and/or as therapeutics for various diseases, disorders, and conditions.

The term “Very Long Chain Polyunsaturated Fatty Acid”, as used herein, generally refers to any polyunsaturated fatty acid or source thereof having at least 28 carbon atoms per chain and having 3 or more carbon:carbon double bonds, including but not limited to, n3 (ω-3) and n6 (ω-6) polyunsaturated fatty acids.

More particularly, the PUFAs referred to herein may be polyunsaturated fatty acids with a carbon chain length of at least 28 carbons (such as but not limited to at least 30, at least 32, at least 34, at least 36, or at least 38 carbons) or even 40 carbons, and the PUFAs may have at least 3 or more double bonds (such as but not limited to, 4 or more, 5 or more, or 6 or more double bonds), wherein all double bonds are typically in the cis configuration. In some embodiments, the presently disclosed and claimed inventive concept(s) comprises methods of producing VLC-PUFAs and VLC-PUFA compositions produced therefrom; in certain embodiments, the compositions are produced recombinantly.

The compositions of some embodiments of the presently disclosed and claimed inventive concept(s) comprise, in various non-limiting embodiments, a dietary supplement, a food product, a nutritional formulation, a pharmaceutical formulation, a humanized animal milk, an infant formula, and a cosmetic item, for example. A formulation of some embodiments of the presently disclosed and claimed inventive concept(s) can be used as a treatment for at least one of, but not limited to (1) enhancing neural development and function, (2) a neurodegenerative disease, (3) an ocular disorder, (4) a retinal disorder, (5) age related maculopathy, (6) a fertility disorder, particularly regarding sperm or testes, and (7) a skin disorder. In some embodiments, the presently disclosed and claimed inventive concept(s) further relates to processes for production of Very Long Chain Polyunsaturated Fatty Acids (VLC-PUFAs). In some embodiments, the presently disclosed and claimed inventive concept(s) also relates to compositions (e.g., nutritional supplements, food products, and pharmaceutic compositions) containing VLC-PUFAs having chain lengths of C28-C40.

One particular, non-limiting embodiment of the presently disclosed and claimed inventive concept(s) is directed to a method of enhancing fertility in a male or female subject in need of such therapy. In the method, an effective amount of a composition described or otherwise contemplated herein is administered to the male or female subject. For example but not by way of limitation, the composition may be administered to a male subject as a nutritional supplement for enhancing sperm function and/or viability. Alternatively, in a method of enhancing sperm function and/or viability of a male subject, a quantity of a composition described or otherwise contemplated herein is added to a seminal fluid or sperm of the male subject.

Another particular, non-limiting embodiment of the presently disclosed and claimed inventive concept(s) is directed to a method of treating an ocular disorder in a subject in need of such therapy. In this method, an effective amount of a composition described or otherwise contemplated herein is administered to the subject.

A further particular, non-limiting embodiment of the presently disclosed and claimed inventive concept(s) is directed to a method of enhancing neural development and function in a subject. In this method, a nutritional supplement comprising a composition described or otherwise contemplated herein is administered to the subject.

Examples disclosed herein describe and demonstrate specific embodiments within the scope of the presently disclosed and claimed inventive concept(s). The examples are given solely for the purpose of illustration and are not to be construed as limitations of the various embodiments of the presently disclosed and claimed inventive concept(s), as many variations thereof are possible without departing from the spirit and scope of the inventive concept(s). All exemplified amounts are weight percentages based upon the total weight of the composition, unless otherwise specified.

Two main families of polyunsaturated fatty acids (PUFAs) are the ω3 (n3) fatty acids, exemplified by eicosapentaenoic acid (EPA), and the ω6 (n6) fatty acids, exemplified by arachidonic acid (ARA). PUFAs are important components of the plasma membrane of the cell, where they may be found in such forms as phospholipids and triglycerides. PUFAs are necessary for proper development, particularly in the developing infant brain, and for tissue formation and repair. PUFAs also serve as precursors to other molecules of importance in human beings and animals, including the prostacyclins, eicosanoids, leukotrienes and prostaglandins. Four major long chain PUFAs of importance include docosahexaenoic acid (DHA) and EPA, which are primarily found in different types of fish oil, gamma-linolenic acid (GLA), which is found in the seeds of a number of plants, and stearidonic acid (SDA), which is found in marine oils and plant seeds. Both GLA and another important long chain PUFA, arachidonic acid (ARA), are found in filamentous fungi. ARA can be purified from animal tissues including liver and adrenal gland. GLA, ARA, EPA and SDA are themselves, or are dietary precursors to, important long chain fatty acids involved in prostaglandin synthesis, in treatment of heart disease, and in development of brain tissue.

Several disorders respond to treatment with fatty acids. For example, dietary supplementation with PUFAs has been shown to reduce the rate of restenosis after angioplasty. Fish oil supplements have been shown to improve symptoms of inflammation and rheumatoid arthritis, and PUFAs have been suggested as treatments for asthma and psoriasis. Evidence indicates that PUFAs may be involved in calcium metabolism, suggesting that PUFAs may be useful in the treatment or prevention of osteoporosis and of kidney or urinary tract stones.

However, in spite of the evidence of the requirements of VLC-PUFAs for various developmental and physiologic processes, commercially-available dietary supplements, nutritional formulations, and pharmaceutical formulations which contain C28-C40 VLC-PUFAs are not currently available since, prior to the current work, there has been no known method of synthesizing such VLC-PUFAs.

Novel methods are thus provided herein for the production of VLC-PUFAs. Novel expression systems using nucleic acids encoding ELOVL4 protein and/or polypeptides having ELOVL4 elongase activity are described. The methods involve growing a host microorganism, cell, plant, or animal which contains and expresses one or more transgenes encoding ELOVL4 protein and/or a polypeptide having ELOVL4 elongase activity. The methods of the presently disclosed and claimed inventive concept(s), unless otherwise specified, generally result in the production of a mixture of VLC-PUFAs of C28-C40 chain length, although production of specific VLC-PUFAs can be enhanced by exposing the host organism to specific precursor substrates or by the use of host organisms having suites of particular fatty acid synthesizing genes.

Examples of VLC-PUFAs which may be used or manufactured in the presently disclosed and claimed inventive concept(s) for use in any composition described or contemplated herein include all or any combination of the following (but are not limited thereto):
(1) All (C28-C40) n-3 fatty acid families; including but not limited to: 28:4n3, 28:5n3, 28:6n3, 28:7n3, 28:8n3, 30:4n3, 30:5n3, 30:6n3, 30:7n3, 30:8n3, 32:4n3 32:5n3, 32:6n3, 32:7n3, 32:8n3, 34:4n3, 34:5n3, 34:6n3, 34:7n3, 34:8n3, 36:4n3, 36:5n3, 36:6n3, 36:7n3, 36:8n3, 38:4n3, 38:5n3, 38:6n3; 38:7n3, 38:8n3, 40:4n3, 40:5n3, 40:6n3, 40:7n3, and 40:8n3, and
(2) All (C28-C40) n-6 fatty acid families; including but not limited to:

28:3n6, 28:4n6, 28:5n6, 28:6n6, 28:7n6, 30:3n6, 30:4n6, 30:5n6, 30:6n6, 30:7n6, 32:3n6, 32:4n6, 32:5n6, 32:6n6, 32:7n6, 34:3n6, 34:4n6, 34:5n6, 34:6n6, 34:7n6, 36:3n6, 36:4n6, 36:5n6, 36:6n6, 36:7n6, 38:3n6, 38:4n6, 38:5n6, 38:6n6, 38:7n6, 40:3n6, 40:4n6, 40:5n6, 40:6n6, and 40:7n6.

In at least one embodiment, a host cell transfected with a nucleic acid sequence comprising an Elovl4 gene (such as, but not limited to, SEQ ID NO.:1, 3, 5, 7, 9, 11, 13, 15 or 17) is provided. The host cell is able to produce the ELOVL4 protein (such as, but not limited to, SEQ ID NO.:2, 4, 6, 8, 10, 12, 14, 16 or 18) which is used within the transfected host cell to produce a mixture of VLC-PUFAs, such as are described elsewhere herein. In another embodiment, the nucleic acid may be an isolated nucleic acid comprising a sequence which anneals to one or more of nucleotide sequences SEQ ID NO.:1, 3, 5, 7, 9, 11, 13, 15 or 17, and which encodes an amino acid sequence having ELOVL4 activity such as SEQ ID NO.:2, 4, 6, 8, 10, 12, 14, 16 or 18, or proteins homologous thereto which have conservative substitutions and which have ELOVL4 activity. In some embodiments, the presently disclosed and claimed inventive concept(s) also provides a host cell having an isolated nucleic acid sequence able to hybridize to an ELOVL4 nucleotide sequence as described herein and which has at least 71% identity thereto, at least 74% identity thereto, at least 80% identity thereto, at least 83% identity thereto, at least 85% identity thereto, at least 90% identity thereto, at least 92% identity thereto, at least 94% identity thereto, at least 96% identity thereto, at least 98% identity thereto, or at least 99% identity thereto, particularly wherein the ELOVL4 nucleotide sequence to which the sequence identity is compared to is SEQ ID NO:3 (see Table 1). As used herein, unless otherwise specified, reference to a percent (%) identity refers to an evaluation of homology which is performed using software including, but not limited to, a BLAST 2.0 Basic BLAST homology search. The host cell of the presently disclosed and claimed inventive concept(s) may comprise a nucleic acid construct comprising an Elovl4 gene, as described herein, which is operably linked to a promoter which is functional in the host cell. In one embodiment of the present invention, the recombinant Elovl4 nucleic acid molecule of the host cell is operatively linked to at least one transcription control sequence.

TABLE 1
Homologies between SEQ ID NO: 3 and other
ELOVL4 expression systems

| Nucleotide sequences (species) | % Identity |
|---|---|
| SEQ ID NO: 1 (Homo sapiens) | 85 |
| SEQ ID NO: 3 (Mus musculus) | 100 |
| SEQ ID NO: 5 (Macaca) | 84 |
| SEQ ID NO: 7 (Bos taurus) | 84 |
| SEQ ID NO: 9 (Equs caballus) | 83 |
| SEQ ID NO: 11 (Gallus gallus) | 74 |
| SEQ ID NO: 13 (Danio rerio) | 74 |
| SEQ ID NO: 15 (Xenopus) | 71 |
| SEQ ID NO: 17 (Yeast) | |

The host cell or organism is either eukaryotic or prokaryotic. Examples of eukaryotic host cells or organisms are those selected from the group consisting of a mammalian cell, an avian cell, a protozoan, an insect cell, a fungal cell such as a yeast, such as Sacchoromyces sp., and an algae cell, such as a marine algae. In some embodiments prokaryotic cells include those selected from the group consisting of a bacteria, particularly E. coli, a cyanobacteria, cells which contain a bacteriophage, and/or a virus.

The host cells or organisms of the presently disclosed and claimed inventive concept(s) in some embodiments comprise nucleic acids which encode enzymes necessary for production of fatty acid precursors for the production of the C28-C40 VLC-PUFAs such as, but not limited to, 18:3n3, 20:5n3, 22:5n3, 22:6n3, 18:2n6, 20:4n6, 22:4n6, or 22:5n6 or C24 or C26 fatty acids. In at least one embodiment, the host cells synthesize C26 fatty acids as compared to an untransformed host cell which is substantially devoid of a DNA sequence which encodes enzymes necessary for production of C26 fatty acids. Alternatively, the C26 substrate (or other PUFAs<C26) for said polypeptide may be exogenously supplied.

The amino acid sequence of the ELOVL4 protein in certain embodiments is at least 65% identical to at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In other embodiments the amino acid sequence of the ELOVL4 protein is at least 70% identical to at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In other embodiments the amino acid sequence of the ELOVL4 protein is at least 75% identical to at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In other embodiments the amino acid sequence of the ELOVL4 protein is at least 80% identical to at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In other embodiments the amino acid sequence of the ELOVL4 protein is at least 85% identical to at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In other embodiments the amino acid sequence of the ELOVL4 protein is at least 90% identical to at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In other embodiments the amino acid sequence of the ELOVL4 protein is at least 95% identical to at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, or SEQ ID NO:18. In certain embodiments the amino acid sequence of the ELOVL4 protein is at least 65% identical, at least 67% identical, at least 69% identical, at least 74% identical, at least 80% identical, at least 85% identical, at least 87% identical, at least 89% identical, at least 90% identical, at least 92% identical, at least 94% identical, at least 95% identical, at least 96% identical, at least 97% identical, at least 98% identical, or at least 99% identical to SEQ ID NO:4. Specific examples of amino acid sequence homologies to SEQ ID NO:4 are shown in Table 2.

TABLE 2
Homologies between SEQ ID NO: 4 and other
ELOVL4 amino acid sequences

| Amino acid sequence (species) | % Identity |
|---|---|
| SEQ ID NO: 2 (Homo sapiens) | 92 |
| SEQ ID NO: 4 (Mus musculus) | 100 |
| SEQ ID NO: 6 (Macaca) | 92.7 |
| SEQ ID NO: 8 (Bos taurus) | 89.2 |
| SEQ ID NO: 10 (Equs caballus) | 87.9 |
| SEQ ID NO: 12 (Gallus gallus) | 74.0 |
| SEQ ID NO: 14 (Danio rerio) | 69.3 |
| SEQ ID NO: 16 (Xenopus) | 69.1 |
| SEQ ID NO: 18 (Yeast) | |
| Rat (Rattus norvegicus | 97.1 |
| Salmon (Salmon selar) | 67 |
| Pig (Sus scrofa) | 90.7 |

Particular microbial strains used herein, as noted elsewhere herein, may be chosen from the group consisting of: bacteria, algae, fungi, protozoa or protists. In some embodiments these microbes are capable of growth and production of the bioactive compounds containing two or more unsaturated bonds at temperatures greater than about 15° C., at temperatures greater than about 20° C., at temperatures greater than about 25° C., and/or temperatures greater than about 30° C. Microorganisms contemplated as host cells for use in producing the VLC-PUFAs of the presently disclosed and claimed inventive concept(s) include, but are not limited to, those belonging to the genera Mortierella, Conidiobolus, Pythium, Phytophathora, Penicillium, Porphyridium, Coidosporium, Mucor, Fusarium, Aspergillus, Rhodotorula, and Entomophthora, and particularly Porphyridium cruentum, Mortierella elongata, Mortierella exigua, Mortierella hygrophila, Mortierella ramanniana, var. angulispora, and Mortierella alpina, and Mucor circinelloides and Mucor javanicus. Particular fungi which may be used as host cells are of the order Mucorales. For example, the fungus may be of the genus Mortierella, Phycomyce, Blakeslea, or Aspergillus. In one embodiment a yeast is of the genus Pichia, such as Pichia ciferrii and Pichia pastoris. Examples of bacteria include, for example but not by way of limitation, the genus Propionibacterium. Non-limiting examples of algae host cells include a dinoflagellate and/or a host cell that belongs to the genus Crypthecodinium, such as but not limited to, Crypthecodinium cohnii.

The presently disclosed and claimed inventive concept(s) includes in some embodiments a host cell having a DNA sequence which encodes an amino acid sequence having the elongate activity (i.e., having the ability to elongate C26 fatty acids) of an ELOVL4 enzyme, wherein the DNA sequence encoding the amino acid sequence is capable of hybridizing under moderate, high, or very high stringency conditions (described below) to or with a nucleic acid molecule encoding an ELOVL4 protein as contemplated herein, or a complement thereof. The nucleic acid sequence encoding the protein having ELOVL4 activity may hybridize under moderate, high or very high stringency conditions to the nucleic acid sequence comprising any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, or SEQ ID NO:17, or a complement thereof.

As used herein, hybridization conditions refer to standard hybridization conditions under which nucleic acid molecules are used to identify similar nucleic acid molecules. Such standard conditions are disclosed, for example, in Sambrook et al. (Molecular Cloning: A Laboratory Manual, Cold Spring Harbor Labs Press, 1989; incorporated by reference herein in its entirety—see specifically, pages 9.31-9.62 thereof).

In certain embodiments, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% to 95% nucleic acid sequence identity with the nucleic acid molecule being used to probe in the hybridization reaction (i.e., conditions permitting about 10% to 5% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C., less than for DNA:RNA hybrids.

In certain embodiments, stringent hybridization conditions for DNA:DNA hybrids include hybridization at an ionic strength of 6×SSC (0.9 M Na⁺) at a temperature of between about 20° C. and about 35° C. (lower stringency), such as but not limited to between about 28° C. and about 40° C. (more stringent) and between about 35° C. and about 45° C. (even more stringent) with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., such as but not limited to between about 38° C. and about 50° C., and between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash at about 37° C. in about 0.1×-0.5×SSC, followed by at least one wash at about 68° C. in about 0.1×-0.5×SSC).

As used herein, a genetically modified plant may be used to produce VLC-PUFAs of the presently disclosed and claimed inventive concept(s) and can include any genetically modified plant including higher plants and particularly, any consumable plants or plants useful for producing a desired VLC-PUFA of the presently disclosed and claimed inventive concept(s). Such a genetically modified plant has a genome which is modified (i.e., mutated or changed) from its normal (i.e., wild-type or naturally occurring) form such that the desired result is achieved (i.e., VLC-PUFA production). Genetic modification of a plant can be accomplished using conventional molecular genetic techniques. Methods for producing a transgenic plant, wherein a recombinant nucleic acid molecule encoding a desired amino acid sequence is incorporated into the genome of the plant, are known in the art. In one embodiment, a plant which can be genetically modified according to the presently disclosed and claimed inventive concept(s) is a plant suitable for consumption by animals, including humans.

Plants which can be genetically modified according to the presently disclosed and claimed inventive concept(s) (i.e., plant host cells) include, but are not limited to any higher plants, e.g., consumable plants, including crop plants and especially plants used for their oils. Such plants can include, for example: canola, soybeans, rapeseed, linseed, corn, safflowers, sunflowers and tobacco. Other plants include those plants that are known to produce compounds used as pharmaceutical agents, flavoring agents, neutraceutical agents, functional food ingredients, or cosmetically active agents or plants that are genetically engineered to produce these compounds/agents.

Non-limiting examples of oil seed plants which could be transfected with an Elovl4 gene as contemplated herein and methods of their transfection are described for example in U.S. Pat. No. 7,179,647, the contents of which are expressly incorporated herein in their entirety.

As described above, in at least one embodiment of the presently disclosed and claimed inventive concept(s), a genetically modified prokaryotic microorganism, eukaryotic microbe, or plant includes a microorganism or plant which has an enhanced ability to synthesize VLC-PUFA molecules (products) or which has a newly introduced ability to synthesize specific VLC-PUFAs. According to the presently disclosed and claimed inventive concept(s), “an enhanced ability to synthesize” a product refers to any enhancement, or up-regulation, in a pathway related to the synthesis of the product such that the microorganism or plant produces an increased amount of the VLC-PUFA (including any production of the product where there was none before) as compared to the wild-type microorganism or plant, cultured or grown, under the same conditions. Methods to produce such genetically modified organisms have been described in detail elsewhere herein and in the art.

In some embodiments of the method of production of VLC-PUFAs of the presently disclosed and claimed inventive concept(s), the genetically modified host cells or organisms are cultured or grown in a suitable medium, under conditions effective to produce the VLC-PUFAs. An appropriate, or effective, medium refers to any medium in which a genetically modified host cell or organism (also referred to herein as a microorganism) of the presently disclosed and claimed inventive concept(s), when cultured, is capable of producing the VLC-PUFAs. Such a medium is typically an aqueous medium comprising assimilable carbon, nitrogen, and phosphate sources. Such a medium can also include appropriate salts, minerals, metals and other nutrients and precursor fatty acids. Microorganisms of the presently disclosed and claimed inventive concept(s) can be cultured in conventional fermentation or growth bioreactors. The microorganisms can be cultured by any fermentation process which includes, but is not limited to, batch, fed-batch, cell recycle, and continuous fermentation. In certain embodiments growth conditions for potential host microorganisms according to the presently disclosed and claimed inventive concept(s) are well known in the art. The VLC-PUFAs produced by the genetically modified microorganism can be recovered from the fermentation medium using conventional separation and purification techniques as described elsewhere herein. For example, the fermentation medium can be filtered or centrifuged to remove microorganisms, cell debris, and other particulate matter, and the product can be recovered from the cell-free supernatant by conventional methods, such as, for example but not by way of limitation, ion exchange, chromatography, extraction, solvent extraction, membrane separation, electrodialysis, reverse osmosis, distillation, chemical derivatization, and crystallization. Alternatively, microorganisms producing the VLC-PUFAs, or extracts and various fractions thereof, can be used without removal of the microorganism components from the product.

In some embodiments of the method for production of desired bioactive compounds of the presently disclosed and claimed inventive concept(s), a genetically modified plant is cultured in a fermentation medium or grown in a suitable medium (such as but not limited to, soil). An appropriate, or effective, fermentation medium has been discussed in detail elsewhere herein. A suitable growth medium for higher plants includes any growth medium for plants, including, but not limited to, soil, sand, any other particulate media that support root growth (e.g., vermiculite, perlite, etc.) or hydroponic culture, as well as suitable light, water, and nutritional supplements which optimize the growth of the higher plant. The genetically modified plants of the presently disclosed and claimed inventive concept(s) are engineered to produce significant quantities of the VLC-PUFAs. The compounds can be recovered through purification processes which extract the compounds from the plant. In at least one embodiment, the compound is recovered by harvesting the plant. In this embodiment, the plant can be consumed in its natural state or further processed into consumable products.

In some embodiments, VLC-PUFAs of the presently disclosed and claimed inventive concept(s) are produced by the genetically modified host cell or organism in a dry weight amount of the cell or organism that is at least one of 0.1% to 1%, 1% to 2%, 2% to 3%, 3% to 4%, 4% to 5%, 5% to 10%, 10% to 15%, 15% to 20%, 20% to 25%, 25% to 30%, 30% to 40%, 40% to 50%, 50% to 60%, 60% to 75%, or greater. Yet another embodiment of the presently disclosed and claimed inventive concept(s) relates to a method to produce a humanized animal milk product. This method includes the steps of genetically modifying milk-producing cells of a milk-producing animal with at least one recombinant nucleic acid molecule comprising a nucleic acid sequence encoding at least one biologically active domain of an ELOVL4 protein.

Methods to genetically modify a host cell and to produce a genetically modified non-human, milk-producing animal, are known in the art. Examples of host animals to modify include, but are not limited to, cattle, sheep, pigs, goats, and yaks, which are amenable to genetic manipulation and cloning for rapid expansion of a transgene expressing population. For animals, Elovl4 transgenes can be adapted for expression in target organelles, tissues and body fluids through modification of the gene regulatory regions. Of particular interest is the production of VLC-PUFAs in the breast milk of the host animal.

Viral constructs from Adeno-associated virus (AAV), lentiviral vectors and retroviral vectors with different kinds of promoters can be used to express ELOVL4 in the host cells described herein. The host cells described herein can be transiently or stably transfected with expression vectors with different promoters to drive the expression of the ELOVL4 protein. ELOVL4 can be over-expressed in the host cells contemplated herein as recombinant or pure proteins that can be secreted or purified as pure or substantially pure ELOVL4 proteins. The pure or substantially pure proteins can be used with cell-free or tissue-free homogenates or in combination with other ELOVLs to make the VLC-PUFAs in the presence of C18-C26 n-3 or n-6 fatty acid precursors, such as but not limited to, 18:3n3, 20:5n3, 22:5n3, 22:6n3, 18:2n6, 18:3n6, 20:4n6, 22:4n6, 22:5n6, 24:5n3, 24:6n3, 24:4n6, 26:5n3, 26:6n3, 26:7n3, 26:5n6, or 26:6n6 or other described herein. Generally, n3 precursor substrates (such as 18:3n3, 20:5n3 or 22:5n3) will substantially lead to n3 products, and n6 substrates (e.g., 18:2n6, 20:4n6) will substantially lead to n6 products. In order to custom tailor production of specific VLC-PUFAs in the host cell or organism, a specific combination of fatty acid precursors (as described herein) and fatty acid metabolism enzymes (other than ELOVL4) must be present. Such enzymes for elongation of VLC fatty acids are known in the art.

As noted above, examples of DNA sequences for the Elovl4 gene from different species that could be used in host cells for making the n-3 or n-6 VLC-PUFAs (Supranenes, Supraenoics) of the presently disclosed and claimed inventive concept(s) include, but are not limited to, SEQ ID NOs:1, 3, 5, 7, 9, 11, 13, 15, and 17. Protein sequences encoded thereby include SEQ ID NOs:2, 4, 6, 8, 10, 12, 14, 16, and 18, respectively.

In some embodiments of the presently disclosed and claimed inventive concept(s), the Elovl4 genes (including any appropriate or effective Elovl4 gene not shown herein) can be transduced, transfected or infected into host cells for production of C28-C40 PUFAs. Examples of host cells and organisms that can be genetically engineered to be used to produce the fatty acids in the methods of the presently disclosed and claimed inventive concept(s) include, but are not limited to, those described for example in U.S. Pat. Nos. 5,407,957; 5,492,938; 5,550,156; 5,711,983; 6,027,900; 6,812,009; 6,977,167; 7,001,772; 7,011,962; 7,022,512; 7,179,647; 7,195,791; 7,252,979; and 7,256,022. In certain embodiments, each host cell or organism used in the presently disclosed and claimed inventive concept(s) is capable of endogenously producing C18, C20, C22, C24, and/or C26 fatty acid precursors (saturated or unsaturated) for production of the C28-C40 fatty acids of the presently disclosed and claimed inventive concept(s).

Recovery of the VLC-PUFAs recombinantly produced in the host organisms can be accomplished by any suitable method, including numerous methods known in the art. For example, recovery can include the following method. Harvested cells (fresh or dried) can be ruptured using techniques known to those in the art. Lipids can then be extracted from the cells by any suitable means, such as by supercritical fluid extraction, or by extraction with solvents such as chloroform, hexane, methylene chloride, methanol, isopropanol, ethyl acetate, and the like, and the extract evaporated under reduced pressure to produce a sample of concentrated lipid material. The VLC-PUFAs can be further separated from other lipids by chilling a fatty acid composition such that the saturated fatty acids in the composition precipitate out while the PUFAs remain in solution. The lipids can then be recovered from the extract.

The host organisms used to produce the VLC-PUFAs herein can also be broken or lysed and the lipids recovered into edible oil using standard methods known in the art. The recovered oils can be refined by well-known processes routinely employed to refine vegetable oils (e.g., chemical or physical refining). These refining processes remove impurities from recovered oils before they are used or sold as edible oils. The refining process consists of a series of processes to degum, bleach, filter, deodorize and polish the recovered oils. After refining, the oils can be used directly as a feed or food additive to produce VLC-PUFA-enriched products. Alternatively, the oil can be further processed and purified as outlined herein and then used in the applications as described herein. The purified oil extract of the presently disclosed and claimed inventive concept(s) comprises, in some embodiments, a VLC-PUFA content of 0.1-99%, for example, >5%, >10%, >15%, >20%, >25%, >50%, >60%, >75%, >85%, >90%, 92%, 94%, 95%, 96%, 97%, 98%, or at least 99%. Individual VLC-PUFAs (e.g., 28-40:5n3, 28-40:6n3, 28-40:7n3, 28-40:8n3, 28-40:4n6, 28-40:5n6, 28-40:6n6, or 28-40:7n6) can be obtained in at least 50% purity or greater from a mixture of VLC-PUFAs. They can be further isolated and purified by selective metabolism as well as known chromatographic procedures.

As noted above, the presently disclosed and claimed inventive concept(s) also relates to methods of isolating the VLC-PUFA-containing oil from host cell biomass, wherein the host biomass can be pretreated before extraction of the oil. Due to the relatively mild conditions of the pretreatment process, the thermo- and oxidation-sensitive PUFAs present in the oil may not be exposed to conditions causing degradation.

Thus, in at least one embodiment of the present invention, a process is provided for obtaining an oil comprising at least one VLC-PUFA from a host cell biomass (comprising organisms that have produced the VLC-PUFA), the process comprising:
a) providing, or obtaining, a biomass with a dry matter content of, for example, from 20 to 90%;
b) granulating the biomass into granular particles;
c) drying the granular particles to give dried granules; and
d) extracting or isolating the oil from the dried granules.

In at least one embodiment, the particulate granular form has an average dry matter content of from 30% to 70%. The dried granules resulting from (c) may particularly have an average dry matter content of at least 80%.

Another embodiment of the presently disclosed and claimed inventive concept(s) is directed to a process for the isolation of one or more VLC-PUFA compounds from a host cell biomass, the process comprising: (a) culturing the host cells, e.g., microorganisms, in a fermentation broth under conditions whereby the compound is produced (by the host cells); (b) pasteurizing either the fermentation broth or a host cell biomass derived from the broth; and (c) extracting, isolating or recovering the VLC-PUFAs from the host cell biomass. The pasteurization in (b) is intended to at least partially inactivate one or more PUFA-degrading substance(s) that may be present in the biomass or broth. Such substances can include proteins, such as enzymes (e.g., proteases). In particular, one is seeking to at least partially inactivate lipases, phospholipases and/or lipoxygenases. This pasteurization may take place before granulating (or crumbling or kneading). Suitably, pasteurization is performed on the fermentation broth, although it can be performed on the host cell biomass obtained from the broth. By pasteurization, it is thought that at least some of the substances that can cause degradation of the compound (VLC-PUFA) can be avoided. This pasteurization may at least contribute to the high quality VLC-PUFAs that can be obtained by the presently disclosed and claimed inventive concept(s).

After pasteurization, but before extraction in (c), one may perform granulating (to give granular particles) and drying the granular particles as described above in stages (b) and (c) in another aspect of the presently disclosed and claimed inventive concept(s). Features of one aspect of the presently disclosed and claimed inventive concept(s) are equally applicable, where appropriate, to other aspects.

In one embodiment of a process of the presently disclosed and claimed inventive concept(s), host cell microorganisms are first grown or fermented under conditions that allow production of the VLC-PUFAs to occur. Such fermentation processes are well known in the art: the microorganism is usually fed with a carbon and nitrogen source, along with a number of additional chemicals or substances such as PUFA precursors that allow growth of the microorganism and/or production of the VLC-PUFA. The resulting material from fermentation (which is often called the broth) can then be filtered, or otherwise treated to remove at least part of the aqueous component. Suitably a large proportion of the water is removed, in order to obtain a biomass cake. The biomass at this stage typically has a dry matter content of from 25% to 80%. The biomass can then be granulated into granular particles. This may be achieved by extrusion. However, whichever technique for granulation is chosen, it is desirable that cell disruption is either prevented or minimized. The granular particles can then be dried. The granules can significantly increase the efficiency of the subsequent drying step. The resulting (dried) granules are then particularly suitable for immersion or percolation extraction. The particle sizes of the granules can be adjusted for optimal drying and extraction additions.

In certain embodiments, the VLC-PUFAs are extracted from the dried granules using a solvent. Any suitable solvent known to a person skilled in the art can be employed. However, suitably a non-polar solvent is used, for example, hexane. It is also possible to use solvents in a super critical state, for example, liquid carbon dioxide.

The process of the presently disclosed and claimed inventive concept(s) can enable a cost effective and efficient extraction of the VLC-PUFA oil, and provide an oil of a particularly high quality. For example, the dried granular form (of the host cell biomass) allows one to use the percolation extraction process, which is particularly efficient. In addition, the granules allow the use of a relatively low temperature for extraction, which does not necessarily decrease the yield of the VLC-PUFAs. Furthermore, the dried granules may require reduced amounts of solvent for the extraction process. An additional advantage is that the release of the used solvent from the biomass can be achieved more efficiently (this process is often referred to as desolventising toasting). All combinations of method or process steps as used herein can be performed in any order, unless otherwise specified or clearly implied to the contrary by the context in which the referenced combination is made.

In certain embodiments, strains of host microorganisms, cells, plants or animals used herein are capable of producing a total VLC-PUFA content of at least 0.1% to 1%, 1% to 2%, 2% to 3%, 3% to 4%, about 4% to 5% of dry weight of the product derived therefrom, or at least about 10% of dry weight, or at least about 20% of dry weight. In some embodiments, strains of host microorganisms produce at least about 30% dry weight of VLC-PUFAs, or at least about 40% dry weight of VLC-PUFAs, or at least about 50% dry weight of VLC-PUFAs.

In one embodiment of the presently disclosed and claimed inventive concept(s), an oil recovered, such as by extraction, from the host organism contains at least about 20% dry weight of at least one type of VLC-PUFA contemplated herein. The biomass from which the oil is obtained can comprise, or originate from, any type of host organism able to produce a VLC-PUFA—containing oil, for example, a bacterium, a yeast, a fungus, an algae (or a mixture thereof), an animal cell, or a plant cell.

In some embodiments, the VLC-PUFA compositions of the presently disclosed and claimed inventive concept(s) as produced herein can be used, for example, as dietary substitutes, as supplements of infant formulas, for patients undergoing intravenous feeding, or for preventing or treating malnutrition. For dietary supplementation, the purified VLC-PUFAs may be incorporated into cooking oils, fats or margarines formulated so that in normal use the recipient would receive a useful amount. The VLC-PUFAs may be used as, or in, pharmaceutical compositions.

In some embodiments, the composition of the presently disclosed and claimed inventive concept(s) may comprise a “food product” which refers to any product to be fed to a human or animals, including, for example, food materials to be consumed by humans including but not limited to infant formula and baby food. Food materials to be consumed by domestic pets include but are not limited to dog and cat foods. The food product may be a livestock feed. The food product of the presently disclosed and claimed inventive concept(s) may comprise host organism biomass or the oil extracted therefrom. As described elsewhere herein, the VLC-PUFAs recovered from the biomass of the host organisms of the presently disclosed and claimed inventive concept(s) can be combined with any animal food material, particularly food materials for humans or animals, or can be used alone as a therapeutic composition.

As noted elsewhere herein, the VLC-PUFAs of the presently disclosed and claimed inventive concept(s) can be added to foods for infants, such as infant formula and baby food. According to the presently disclosed and claimed inventive concept(s), an infant refers to infants in utero and to children less than about two years old, including, in particular, premature infants. In some embodiments, the VLC-PUFA produced as described herein is used in a product comprising at least one of a food, a dietary supplement, a pharmaceutical formulation, a humanized animal milk, and an infant formula. In some embodiments the VLC-PUFAs of the presently disclosed and claimed inventive concept(s) can be used in soaps, cosmetics, lotions, creams, oils, shampoos, and similar items produced by the cosmetic and skin care industry. The VLC-PUFAs of the presently disclosed and claimed inventive concept(s) may be formulated into the composition as the free fatty acid or as a salt of the free fatty acid or as compounds or materials that can otherwise provide a source of such free fatty acids upon or following administration to the person, including phospholipids and glyceride esters (mono-, di-, tri-) of the polyunsaturated fatty acids. The compositions of the presently disclosed and claimed inventive concept(s) comprise one or more of the C28-C40 PUFAs described herein, or combinations thereof, alone or in further combination with other PUFAs such as linoleic acid, linolenic acid, ARA, DHA, DPA, and/or EPA. The concentration of C28-C40 VLC-PUFAs in the dietary and supplemental formulas of the presently disclosed and claimed inventive concept(s) includes any concentration or amount which is safe for internal, topical, or commercial use.

The presently disclosed and claimed inventive concept(s) includes in some embodiments an oil comprising one or more C28-C40 VLC-PUFAs. The oil may be an extracted mixture of VLC-PUFAs comprising for example 0.1-99.9% of a C28 PUFA, and/or 0.1-99.9% of a C30 PUFA, and/or 0.1-99.9% of a C32 PUFA, and/or 0.1-99.9% of a C34 PUFA, and/or 0.1-99.9% of a C36 PUFA, and/or 0.1-99.9% of a C38 PUFA, and/or 0.1-99.9% of a C40 PUFA. In one embodiment, the oil extract mixture is purified such that a C28-C40 PUFA mixture comprises at least 1%, at least 5%, at least 25%, or at least 50%, at least 75%, or at least 95% of the total fatty acid content of the oil or mixture of PUFAs.

In one embodiment, a mixture of VLC-PUFAs derived from the host cell or microorganism contains 0.1% to 99.9% of a C28:4n3, 5n3, 6n3, 7n3, 8n3, 3n6, 4n6, 5n6, 6n6, and/or 7n6; and/or 0.1% to 99.9% of a C30:4n3, 5n3, 6n3, 7n3, 8n3, 3n6, 4n6, 5n6, 6n6, and/or 7n6; and/or 0.1% to 99.9% of a C32:4n3, 5n3, 6n3, 7n3, 8n3, 3n6, 4n6, 5n6, 6n6, and/or 7n6; and/or 0.1% to 99.9% of a C34:4n3, 5n3, 6n3, 7n3, 8n3, 3n6, 4n6, 5n6, 6n6, and/or 7n6; and/or 0.1% to 99.9% of a C36:4n3, 5n3, 6n3, 7n3, 8n3, 3n6, 4n6, 5n6, 6n6, and/or 7n6; and/or 0.1% to 99.9% of a C38:4n3, 5n3, 6n3, 7n3, 8n3, 3n6, 4n6, 5n6, 6n6, and/or 7n6; and/or 0.1% to 99.9% of a C40:4n3, 5n3, 6n3, 7n3, 8n3, 3n6, 4n6, 5n6, 6n6, and/or 7n6 PUFA.

Another embodiment of the presently disclosed and claimed inventive concept(s) is a pharmaceutical supplement or composition comprising one or more of the C28-C40 PUFAs described herein in a pharmaceutically acceptable carrier. Further contemplated within the scope of the presently disclosed and claimed inventive concept(s) is a nutritional supplement or other composition that comprises one or more of the C28-C40 PUFAs of the presently disclosed and claimed inventive concept(s). Nutritional and/or pharmaceutical supplements or compositions of the presently disclosed and claimed inventive concept(s) may comprise at least 0.00001%, at least 0.0001%, at least 0.001%, at least 0.01%, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, at least 90%, or at least 95%, by weight, of the supplement or composition.

The nutritional composition of the presently disclosed and claimed inventive concept(s) may be administered to a mammalian subject orally, parenterally or otherwise internally. One composition for internal consumption is an infant formula or an oral dosage form for treatment or prevention of a disease condition. In one embodiment, the nutritional compositions are in a liquid form or a solid form as discussed elsewhere herein. In one non-limiting embodiment of a dietary supplement, the VLC-PUFAs are dissolved in a vegetable oil and supplied, for example, at 1 to 1000 mg of VLC-PUFA per day, or 10-750 mg of VLC-PUFA per day, or 50-500 mg VLC-PUFA per day. For commercial sale for research or other purposes, specific VLC-PUFAs can be supplied as a substantially pure substance (e.g., 99% pure) sealed under an inert gas such as nitrogen or argon and sold in amounts, for example, ranging from 1 mg to 1 gm, as free fatty acids, free fatty acid salts, or in whole lipid form.

In some embodiments of infant formulas of the presently disclosed and claimed inventive concept(s), the formulas typically contain proteins, carbohydrates, lipids, vitamins, minerals, and other nutrients, and are commercially available as reconstitutable powders, ready-to-feed liquids, and dilutable liquid concentrates.

Many nutritional formulas, especially infant formulas, commonly contain a variety of mid-chain and shorter chain PUFAs as part of the lipid component of the overall nutrient system, examples of which include linoleic acid, alpha-linolenic acid, eicosapentaenoic acid (EPA), arachidonic acid (ARA), docosahexaenoic acid (DHA), among others. Arachidonic and docosahexaenoic acids in particular are commonly found in many commercially available infant formulas such as ENFAMIL, ISOMIL, SIMILAC, and ADVANCE.

As such, certain embodiments of the presently disclosed and claimed inventive concept(s) are directed to methods of providing nutrition to an infant, toddler, child, or adult, said methods comprising the administration or feeding a composition of the presently disclosed and claimed inventive concept(s) to the infant, toddler, child, or adult as a nutritional formula or supplement as their sole, primary, or partial nutritional needs.

The compositions of the presently disclosed and claimed inventive concept(s) may comprise protein, carbohydrate, and lipids, as well as the one or more VLC-PUFAs of the presently disclosed and claimed inventive concept(s), and may contain combinations of antioxidant carotenoids including lutein, lycopene, and beta-carotene. These and other elements of the nutritional formulas and corresponding methods are described elsewhere herein and in the art. The compositions may further comprise vitamins, minerals, and electrolytes, and may potentially serve as the sole source of nutrition when provided in sufficient quantity.

It has been shown that infants benefit from consuming breast milk or formula containing docosahexaenoic acid (DHA; 22:6n3) and arachidonic acid (AA; 20:4n6) and related polyunsaturated fatty acids³². As a result, infant formula is now supplemented with such fatty acids. The brain and retina are two areas where VLC-PUFAs are found. Consequently, in one embodiment, the presently disclosed and claimed inventive concept(s) include dietary supplementation to infants and pregnant females with these VLC-PUFAs for aiding in neural development and function in infants.

When the composition is an adult formula, the protein component may comprise, for example, from about 10% to about 80% of the total caloric content of said nutritional formula; the carbohydrate component may comprise, for example, from about 10% to about 70% of the total caloric content of said nutritional formula; and the lipid component may comprise, for example, from about 5% to about 50% of the total caloric content of said nutritional formula. The nutritional formula may be in liquid or powder form. These ranges are provided as examples only, and are not intended to be limiting.

When the composition is a non-adult nutritional composition, the non-adult composition includes those embodiments in which the protein component may comprise for example from about 7.5% to about 25% of the total caloric content of the nutritional composition; the carbohydrate component may comprise for example from about 35% to about 50% of the total caloric content of the nutritional formula; and the lipid component may comprise for example from about 30% to about 60% of the total caloric content of the nutritional formula. These ranges are provided as examples only, and are not intended to be limiting.

Many different sources and types of carbohydrates, lipids, proteins, minerals and vitamins are known and can be used in the nutritional compositions of the presently disclosed and claimed inventive concept(s), provided that such nutrients are compatible with the added ingredients in the selected formula, are safe for their intended use, and do not otherwise unduly impair product performance.

Carbohydrates suitable for use in the nutritional compositions of the presently disclosed and claimed inventive concept(s) can be simple or complex, lactose-containing or lactose-free, or combinations thereof, non-limiting examples of which include hydrolyzed, intact, naturally and/or chemically modified cornstarch, maltodextrin, glucose polymers, sucrose, corn syrup, corn syrup solids, rice or potato derived carbohydrate, glucose, fructose, lactose, high fructose corn syrup and indigestible oligosaccharides such as fructooligosaccharides (FOS), and combinations thereof.

Non-limiting examples of proteins suitable for use in the nutritional compositions include hydrolyzed, partially hydrolyzed or non-hydrolyzed proteins or protein sources, and can be derived from any known or otherwise suitable source such as milk (e.g., casein, whey), animal (e.g., meat, fish), cereal (e.g., rice, corn), vegetable (e.g., soy), or combinations thereof. The proteins for use herein can also include, or be entirely or partially replaced by, free amino acids known for use in nutritional products, non-limiting examples of which include tryptophan, glutamine, tyrosine, methionine, cysteine, arginine, and combinations thereof. Other (non-protein) amino acids typically added to nutritional products include carnitine and taurine. In some cases, the D-forms of the amino acids are considered as nutritionally equivalent to the L-forms, and isomer mixtures are used to lower cost (for example, D,L-methionine).

Non-limiting examples of other lipids suitable for use in the nutritional compositions include coconut oil, soy oil, corn oil, olive oil, safflower oil, high oleic safflower oil, MCT oil (medium chain triglycerides), sunflower oil, high oleic sunflower oil, palm and palm kernel oils, palm olein, canola oil, marine oils, cottonseed oils, linseed oil, flaxseed oil, evening primrose oil, and combinations thereof.

In addition to these food grade oils, structured lipids may be incorporated if desired. Structured lipids are predominantly triacylglycerols containing mixtures of medium and long chain fatty acids on the same glycerol molecule. Structured lipids are described in U.S. Pat. No. 6,160,007, which is also incorporated by reference herein.

The nutritional compositions of the presently disclosed and claimed inventive concept(s) may further comprise any of a variety of vitamins in addition to the carotenoids described hereinbefore, non-limiting examples of which include vitamin A, vitamin D, vitamin E, vitamin K, thiamine, riboflavin, pyridoxine, vitamin B12, niacin, folic acid, pantothenic acid, biotin, vitamin C, choline, chromium, carnitine, inositol, salts and derivatives thereof, and combinations thereof.

The nutritional compositions may further comprise any of a variety of minerals, non-limiting examples of which include calcium, phosphorus, magnesium, iron, zinc, manganese, copper, iodine, sodium, potassium, chloride, selenium, and combinations thereof.

The nutritional compositions of the presently disclosed and claimed inventive concept(s) include embodiments that comprise per 100 kcal of formula one or more of the following: vitamin A (from about 250 to about 750 IU), vitamin D (from about 40 to about 100 IU), vitamin K (greater than about 4 μg), vitamin E (at least about 0.3 IU), vitamin C (at least about 8 mg), thiamine (at least about 8 μg), vitamin B12 (at least about 0.15 μg), niacin (at least about 250 μg), folic acid (at least about 4 μg), pantothenic acid (at least about 300 μg), biotin (at least about 1.5 μg), choline (at least about 7 mg), and inositol (at least about 2 mg).

The nutritional compositions of the presently disclosed and claimed inventive concept(s) include embodiments that comprise per 100 kcal of formula one or more of the following: calcium (at least about 50 mg), phosphorus (at least about 25 mg), magnesium (at least about 6 mg), iron (at least about 0.15 mg), iodine (at least about 5 μg), zinc (at least about 0.5 mg), copper (at least about 60 μg), manganese (at least about 5 μg), sodium (from about 20 to about 60 mg), potassium (from about 80 to about 200 mg), chloride (from about 55 to about 150 mg), and selenium (at least about 0.5 μg).

The nutritional compositions of the presently disclosed and claimed inventive concept(s) may further comprise other optional components that may modify the physical, chemical, aesthetic or processing characteristics of the compositions or serve as pharmaceutical or additional nutritional components when used in the targeted population. Many such optional ingredients are known or otherwise suitable for use in food and nutritional products, including infant formulas, and may also be used in the nutritional compositions of the presently disclosed and claimed inventive concept(s), provided that such optional materials are compatible with the essential materials described herein, are safe for their intended use, and do not otherwise unduly impair product performance.

Non-limiting examples of such optional ingredients include preservatives, additional anti-oxidants, emulsifying agents, buffers, colorants, flavors, nucleotides and nucleosides, probiotics, prebiotics, lactoferrin and related derivatives, thickening agents and stabilizers.

For powder embodiments of compositions of the presently disclosed and claimed inventive concept(s), the above-described methods of use further comprise reconstitution of the powder with a suitable aqueous liquid (such as but not limited to water) followed by oral or enteral administration of the resulting nutritional liquid to provide the person with their sole, primary, or supplemental nutrition. Such dilution may be in an amount sufficient to provide a caloric density appropriate for the patient population to which the formula is directed.

The nutritional formulas of the presently disclosed and claimed inventive concept(s) may have any caloric density suitable for the targeted or intended patient population, or provide such a density upon reconstitution of a powder embodiment or upon dilution of a liquid concentrate embodiment. Most common caloric densities for the infant formulas embodiments are generally at least about 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter). Generally, the 22-24 kcal/fl oz formulas are more commonly used in pre-term or low birth weight infants, and the 20-21 kcal/fl oz (675-680 to 700 kcal/liter) formulas are more often used in term infants. Non-infant and adult nutritional compositions may have any caloric density suitable for the targeted or intended population.

For powder embodiments of the presently disclosed and claimed inventive concept(s), such powders are typically in the form of flowable or substantially flowable particulate compositions, or at least particulate compositions that can be easily scooped and measured with a spoon or similar other device, wherein the compositions can easily be reconstituted by the intended user with a suitable aqueous fluid, typically water, to form a liquid nutritional formula for immediate oral or enteral use. In this context, “immediate” use generally means within about 48 hours, most typically within about 24 hours, and in certain particular embodiments, means right after reconstitution. These powder embodiments include spray dried, agglomerated, dry mixed or other known or otherwise effective particulate form. The quantity of a nutritional powder required to produce a volume suitable for one serving can vary.

The nutritional compositions of the presently disclosed and claimed inventive concept(s) may be packaged and sealed in single or multi-use containers, and then stored under ambient conditions for up to about 36 months or longer, more typically from about 12 to about 24 months. For multi-use containers, these packages can be opened and then covered for repeated use by the ultimate user, provided that the covered package is then stored under ambient conditions (e.g., avoid extreme temperatures) and the contents used within about one month or so.

The nutritional compositions of the presently disclosed and claimed inventive concept(s) may be prepared by any known or otherwise effective technique suitable for making and formulating nutritional compositions or similar other compositions, variations of which may depend upon variables such as the selected product form, ingredient combination, packaging and container selection, and so forth, for the desired nutritional formula. Such techniques and variations for any given formula are easily determined and applied by one of ordinary skill in the nutritional formulation or manufacturing arts.

The nutritional compositions of the presently disclosed and claimed inventive concept(s), including the exemplified formulas described herein, can therefore be prepared by any of a variety of known or otherwise effective formulation or manufacturing methods. These methods most typically involve the initial formation of an aqueous slurry containing the carbohydrates, proteins, lipids, stabilizers or other formulation aids, vitamins, minerals, or combinations thereof. The slurry is emulsified, pasteurized, homogenized, and cooled. Various other solutions, mixtures, or other materials may be added to the resulting emulsion before, during, or after further processing. This emulsion can then be further diluted, heat-treated, and packaged to form a ready-to-feed or concentrated liquid, or it can be heat-treated and subsequently processed and packaged as a reconstitutable powder, e.g., spray dried, dry mixed, or agglomerated. Other suitable methods for making nutritional formulas are described, for example, in U.S. Pat. No. 6,365,218, U.S. Pat. No. 6,589,576, U.S. Pat. No. 6,306,908, U.S. Patent Application 20030118703 A1, the descriptions of which are incorporated herein by reference in their entireties.

As noted, the VLC-PUFAs described herein are also suitable for use as therapeutic (pharmaceutic) and experimental agents. One embodiment of the presently disclosed and claimed inventive concept(s) comprises the use of the VLC-PUFAs for treatment of various deficiencies, disorders and conditions in infants and adults as discussed elsewhere herein. Parenteral routes of administration include, but are not limited to, subcutaneous, intradermal, intravenous, intramuscular and intraperitoneal routes. The presently disclosed and claimed inventive concept(s) includes compositions of VLC-PUFAs and a carrier suitable for therapeutic delivery. As used, herein, a “carrier” refers to any substance suitable as a vehicle for delivering a molecule or composition to a suitable in vivo site of action. Examples of such carriers include, but are not limited to water, phosphate buffered saline (PBS), Ringer's solution, dextrose solution, serum-containing solutions, Hank's solution and other aqueous physiologically-balanced solutions. Acceptable protocols to administer the present compositions in an effective manner include individual dose size, number of doses, frequency of dose administration, and mode of administration. Determination of such protocols can be accomplished by those skilled in the art depending upon a variety of variables known in the art. Other embodiments of the presently disclosed and claimed inventive concept(s) comprise VLC-PUFA compositions for treatment of adults or non-infants.

Acceptable protocols for administration of the VLC-PUFA compositions described herein include parenteral feeding techniques or encapsulating oil recovered from a host organism in a capsule, such as a gelatin (i.e., digestible) capsule, for oral administration and/or in a liquid diet formulation. A liquid diet formulation can comprise a liquid composition containing nutrients suitable for supplementing a diet or nutrients sufficient as a complete diet. For pharmaceutical use (human or veterinary), the VLC-PUFA compositions described herein may be administered orally but can be administered by any route by which they may be successfully absorbed, e.g., parenterally (e.g., subcutaneously, intramuscularly or intravenously), rectally or vaginally or topically, for example, as a skin ointment or lotion. The VLC-PUFAs described herein may be administered alone or in combination with a pharmaceutically acceptable carrier or excipient. Where available, gelatin capsules are a form of oral administration. Dietary supplementation as set forth above also can provide an oral route of administration. The VLC-PUFAs described herein may be administered in conjugated forms, or as salts, esters, triacylglycerols, phospholipids, whole lipids, amides or prodrugs of the fatty acids. Any pharmaceutically acceptable salt is encompassed by the presently disclosed and claimed inventive concept(s); including the sodium, potassium or lithium salts. Esters in one embodiment are ethyl esters. As solid salts, the VLC-PUFAs also can be administered in tablet form. For intravenous administration, the VLC-PUFAs may be incorporated into commercial lipid emulsion formulations such as INTRALIPIDS.

The VLC-PUFA compositions contemplated for use herein may be delivered to the human or animal recipient (e.g., dogs, cats, pigs, sheep, goats, chickens, turkeys, zoo animals, other livestock, horses) via oral dosage forms, such as a capsules, powders, tablets, oils, feed stock, or other supplements, as described for example in U.S. Pat. No. 6,641,837 (Col. 3-4), U.S. Pat. No. 6,652,879 (Col. 1-4), U.S. Pat. No. 7,264,824 (Col. 1-3, and WO 90/04391 and WO 96/36329 cited therein), U.S. Pat. No. 7,112,609 (Col. 12-13), U.S. Pat. No. 5,948,818 (Col. 1-8), U.S. Pat. No. 6,726,924 (Col. 1-6), U.S. 2006/0264409 A1 paragraph 64-90, and all U.S. patents cited therein.

The lipid composition of the spermatozoan membrane may be a major determinant of motility, cold sensitivity and a wide selection of factors associated with overall viability within fresh ejaculates or stored ejaculates maintained at −196° C. for artificial insemination. Accordingly, the composition of the presently disclosed and claimed inventive concept(s) may further comprise an antioxidant to enhance sperm function and/or viability. The nutritional and pharmaceutical compositions described herein may be used to improve male and/or female fertility, particularly in mammalian species such as e.g., cattle, pigs, sheep, humans, dogs, cats, goats, horses, and zoo and livestock animals.

In a further aspect, the presently disclosed and claimed inventive concept(s) includes a method of enhancing sperm function and/or viability, comprising adding to the semen of an animal substantially sperm-free seminal fluid containing a VLC-PUFA, and optionally an antioxidant.

The seminal fluid may be produced from the semen of another animal which may have been vasectomised or from whose semen sperm have been removed. The mixture of the semen and seminal fluid can then be stored at low temperature for use in artificial insemination. The semen in this aspect of the presently disclosed and claimed inventive concept(s) may already have been boosted in function or viability by virtue of the animal having antioxidant and/or other VLC-PUFAs administered to it. In one non-limiting example, the VLC-PUFA is administered to the animal in an amount of, for example, at least 1-1000 mg of VLC-PUFA per kg of body weight, or at least 10-750 mg of VLC-PUFA per kg of body weight, or at least 50-500 mg of VLC-PUFA per kg of body weight.

In certain embodiments, the presently disclosed and claimed inventive concept(s) includes a method of enhancing the function and/or viability of sperm, comprising controlling the VLC-PUFA content of the sperm, for example, in the plasma membrane of the sperm, although the control of VLC-PUFA content of the seminal plasma can also be of benefit.

The presently disclosed and claimed inventive concept(s) also includes a method of combating sperm dysfunction, comprising controlling the VLC-PUFA content of the sperm, for example, of the content of the sperm plasma membrane, e.g., by exposing the sperm to one or more VLC-PUFAs described herein.

The VLC-PUFAs can be added directly to the ejaculate, or can be administered to an animal to enhance the function and/or viability of sperm from that animal. In such a case, the VLC-PUFAs may be particularly administered in quantities of at least 0.1 to 1000 mg/kg body weight. The VLC-PUFAs can be provided in substantially pure form (e.g., 95% or more) or in combination with a pharmaceutical carrier or excipient, or in impure form. The VLC-PUFAs may be incorporated into the fatty acid pool of the sperm, or may remain in the seminal fluid in order to exert its beneficial effects. The viability can be further enhanced by increased mobility, cold resistance or related factors. It has been shown that mice deficient in VLC-PUFA are sterile and possess a lack of mature sperm cells, which is a frequent cause of male infertility in humans³¹. Increasing the content therein of VLC-PUFAs (C28-C40) (which are normally present in sperm cells), will lead to the production of viable, mature sperm cells resulting in restored fertility.

In some embodiments, the presently disclosed and claimed inventive concept(s) further comprises methods of treating subjects that have Stargardt disease, retinal diseases, and macular degeneration (or treating subjects to prevent or delay progression of such diseases) by dietary supplementation with C28-C40 n-3 fatty acids, and/or C28-C40 n-6 fatty acids as described herein, for protecting or slowing down photoreceptor degeneration in these patients.

Stargardt-like macular dystrophies are a group of progressive photoreceptor degenerative disorders that eventually lead to loss of vision. They are distinct from other types of macular degeneration because their phenotype is evident in the early years, even in the first decade, as opposed to age-related macular degeneration (AMD), which is the most common cause of blindness in people over 60 years of age. Three mutations in the Elovl4 gene are known to cause one form of the disease named Stargardt-3 (STGD3). However, the fact that ELOVL4 is indeed necessary for fatty acid elongations and the specific step(s) in the elongation process it catalyzes was not demonstrated prior to the present work.

### EXPERIMENTAL

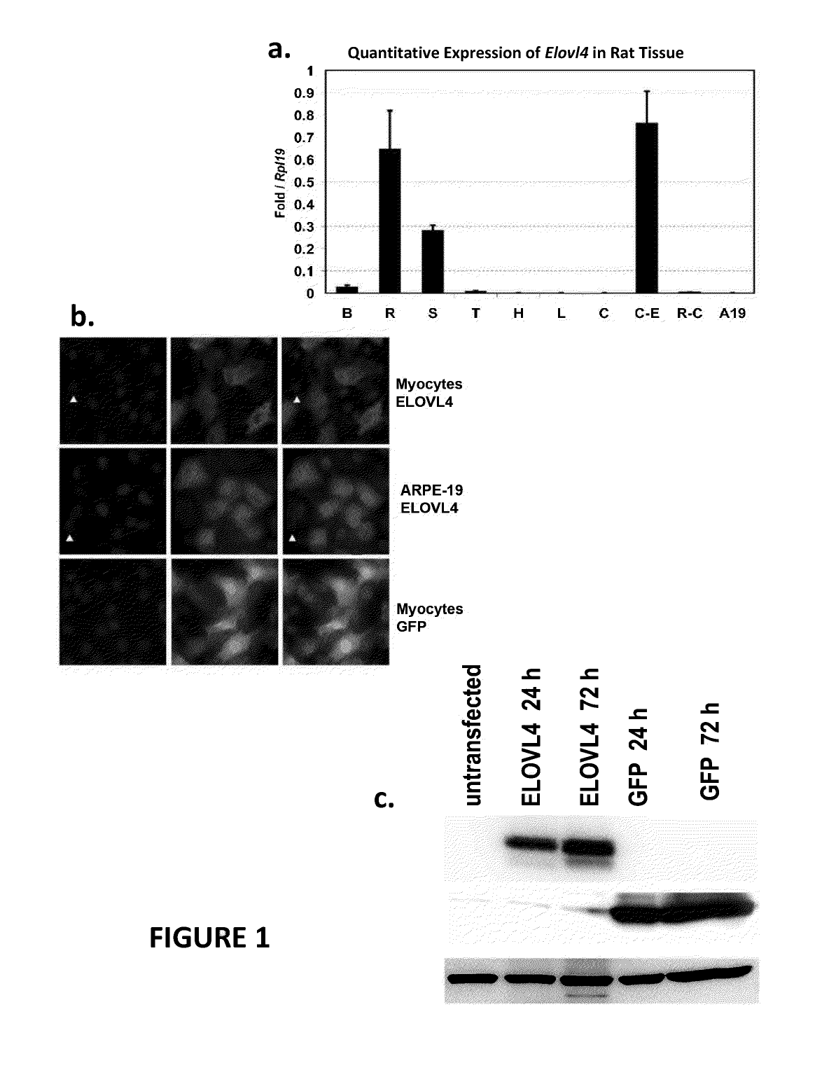

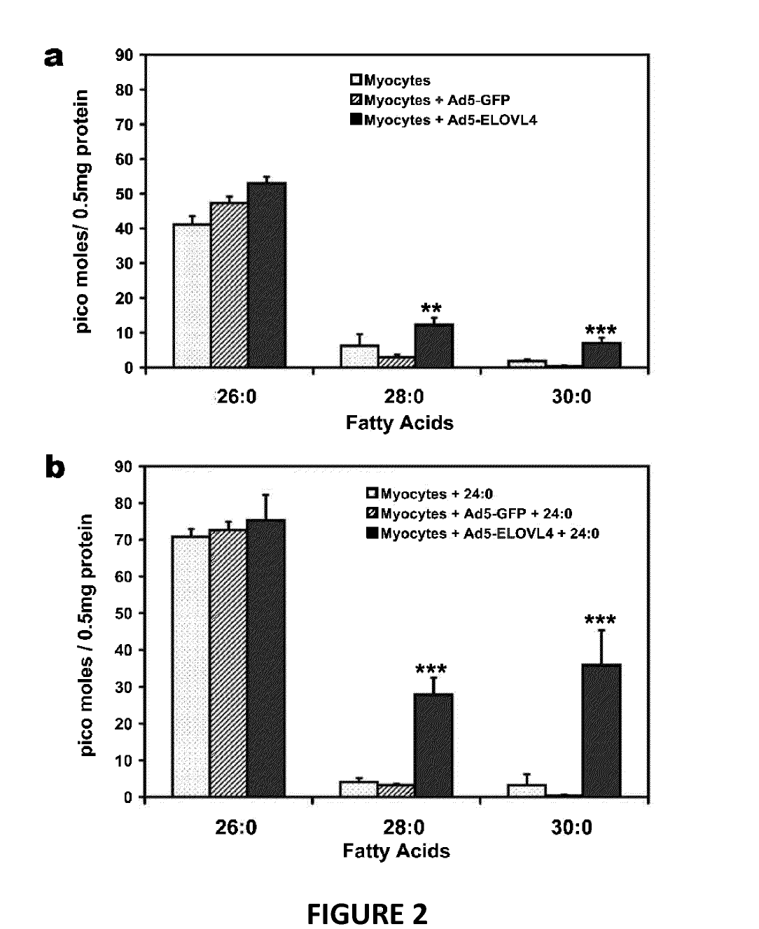

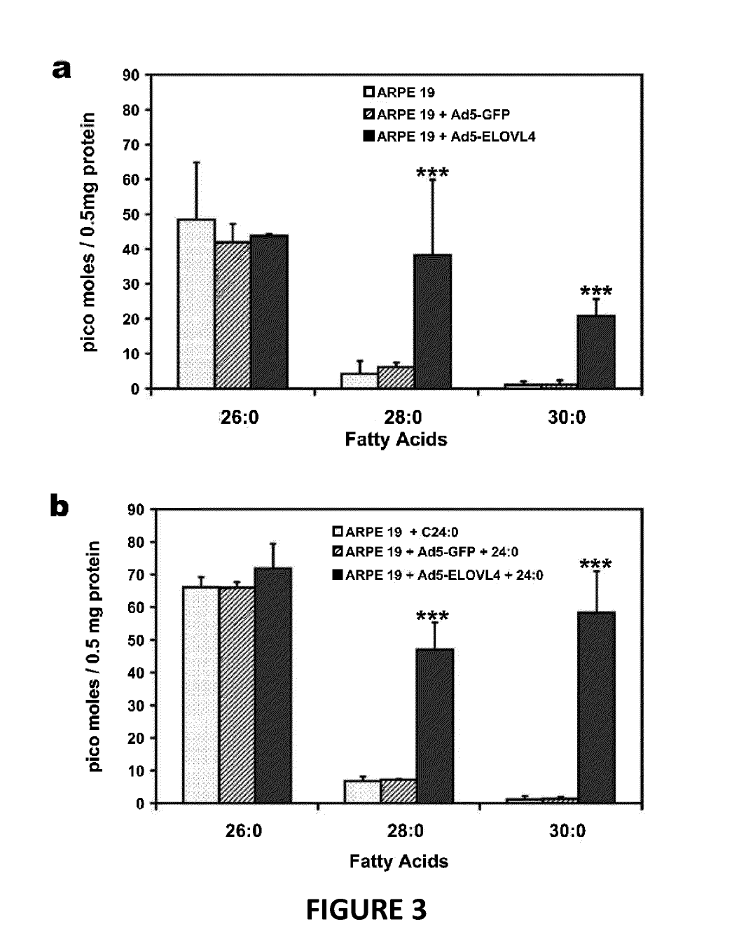

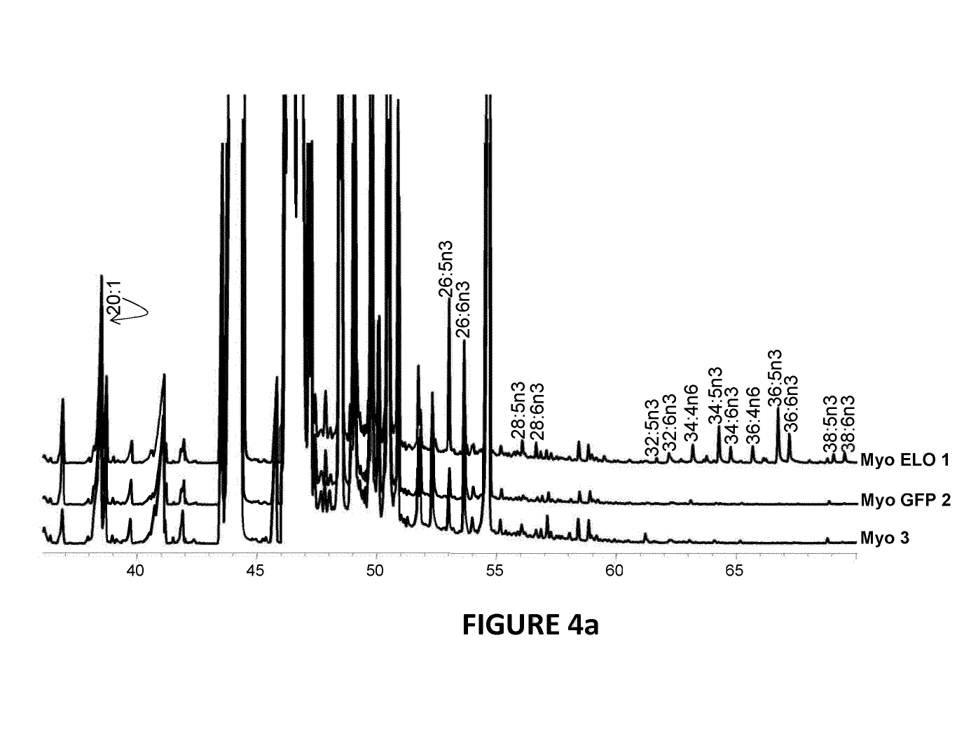

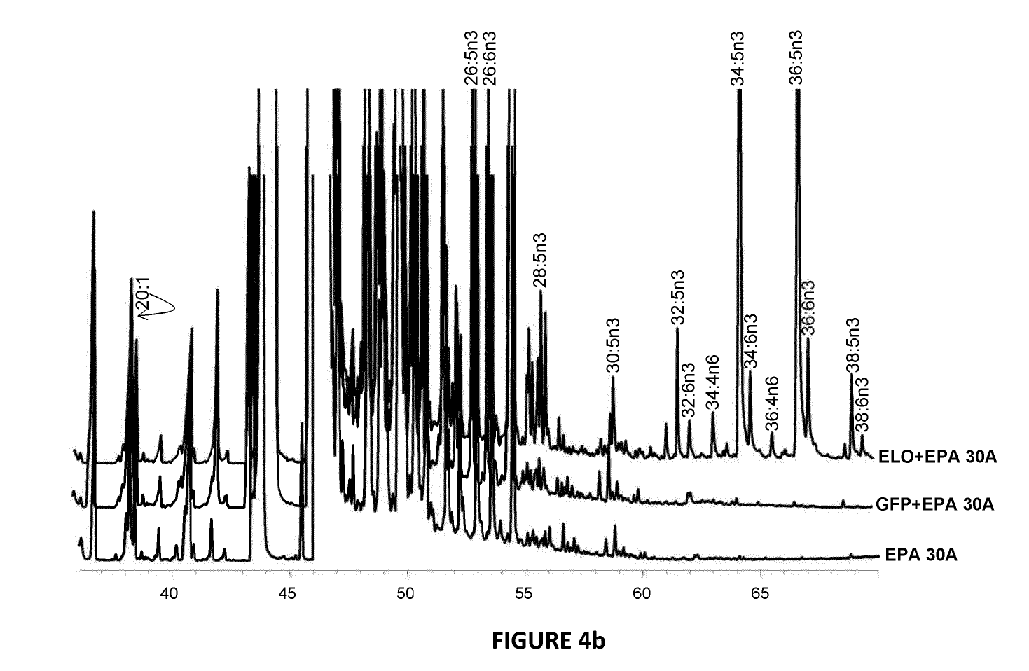

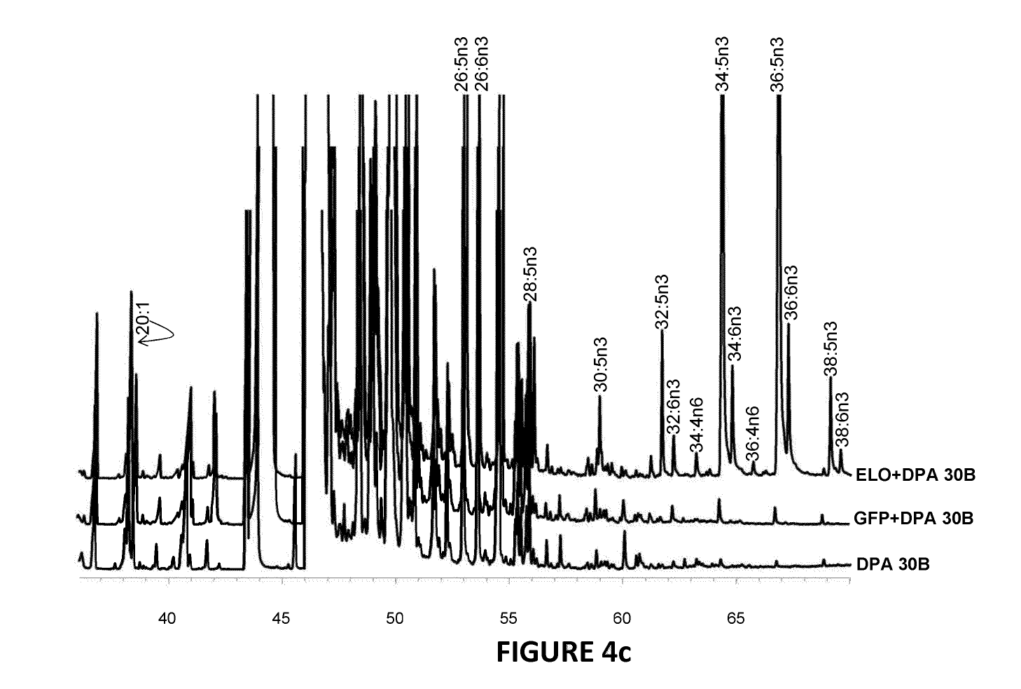

The role of the Elovl4 gene and the protein encoded in the biosynthesis of very long chain fatty acids (VLCFAs) was investigated in rat neonatal cardiac myocytes (FIG. 2) and a human retina epithelium cell line (ARPE-19) (FIG. 3), neither of which expresses ELOVL4 mRNA or protein to any extent (FIG. 1a). It has been demonstrated herein that elongation of fatty acid C26 to fatty acids C28-C40 is enhanced in cells expressing ELOVL4. Adenovirus-mediated expression of ELOVL4 protein in myocytes or ARPE-19 cells resulted in elongation of the precursor tetracosanoic acid/lignoceric acid (24:0) to hexacosanoic acid (26:0) among all treatment groups. However, ELOVL4-expressing cells further elongated 26:0 to octacosanoic acid/montanic acid (28:0) and triacontanoic acid/mellisic acid (30:0) at significantly higher levels than control cells. Likewise, only ELOVL4-expressing cells elongated the polyunsaturated fatty acids eicosapentaenoic acid (20:5n3) (FIG. 4b) and docosapentaenoic acid (22:5n3) (FIG. 4c) to C28-C40 polyunsaturated fatty acids (PUFA). For the first time, direct evidence is provided for the specific step in fatty acid metabolism catalyzed by ELOVL4. The presently disclosed and claimed inventive concept(s) demonstrates that these steps are important in the synthesis of VLC-FA and VLC-PUFA (C28-C40) that are esterified into phosphatidylcholine, sphingomyelin, ceramides and free fatty acids in retina, brain, skin, testis and sperm.

To establish the specific step that the ELOVL4 protein catalyzes in VLCFA elongation, transgenic mouse ELOVL4 protein was overexpressed in rat neonatal cardiomyocytes and in a human retinal pigment epithelium cell line (ARPE-19). These cells have none or low detectable levels of endogenous ELOVL4 protein and mRNA (FIG. 1a). The endogenous expression of ELOVL4 in rat cardiomyocytes cells was ^{˜}2500-fold less than that found in the retina by quantitative real-time PCR (FIG. 1). The highest level of ELOVL4 expression was detected in rat retina, followed by skin; brain and testis also showed significant expression which was ^{˜}1000 times lower than the level found in retina; heart and liver showed negligible levels of expression, corresponding well with previous observations in mice¹⁹. Minimal ELOVL4 expression was observed in ARPE19 cells (FIG. 1a). When the cardiomyocytes and ARPE19 cells were transduced with adenovirus carrying mouse ELOVL4 (Ad5-mELOVL4) and subjected to qRT-PCR, ELOVL4 expression was increased 1000- and 3000-fold, respectively, compared to non-transduced cells. This expression level is comparable to that observed in rat retina (FIG. 1a).

Further, adenoviral delivery and expression of ELOVL4 or GFP proteins in transduced cells were confirmed by immunocytochemical labeling for ELOVL4 and GFP (FIG. 1b). Non-transduced cells did not show any expression of ELOVL4 in both cell types (FIG. 1b, arrows). Finally, protein expression levels in the transduced cells were confirmed by Western blot (FIG. 1c). Mouse ELOVL4 protein was not detected in control cells (non-transduced and Ad5-GFP transduced cells), but was highly abundant in cardiomyocytes (FIG. 1c) and ARPE-19 cells transduced with Ad5-mELOVL4 (data not shown).

It was then tested whether expression of mouse ELOVL4 can lead to elongation of a saturated long chain fatty acid substrate, lignoceric acid (24:0), a precursor of VLCFAs greater than 26 carbons. From the fatty acid data obtained by GC/MS, it was found that the cells, irrespective of ELOVL4 over-expression, were able to internalize the precursors and elongate them to C26 products (FIGS. 2a&b). These elongation steps are possibly catalyzed by other elongates as indicated in previous studies^{9,10}. The fatty acid profiles were, however, remarkably different between the controls (non-transduced and Ad5-GFP transduced) and the Ad5-mELOVL4 transduced cells in terms of elongation past C26. Even in the absence of exogenous 24:0 precursors, the Ad5-mELOVL4-transduced cells showed three-fold increases in the levels of 28:0 and 30:0 compared to controls (FIG. 2a). Upon treatment with 24:0, cardiomyocytes transduced with Ad5-GFP and non-transduced controls had no change in their fatty acid profiles. However, the Ad5-mELOVL4 transduced cells showed significant elongation of the 26:0 fatty acids to 28:0 fatty acids (79.1% increase compared to 11.3% and 9.3% in myocytes and GFP expressing cells, respectively). The 28:0 fatty acid was further elongated to 30:0 (FIG. 2b). Compared to the non-transduced and Ad5-GFP-transduced cells, which showed no differences in the levels of 28:0 and 30:0 fatty acids, the Ad5-mELOVL4-transduced cells showed statistically significant increases in 28:0 and 30:0 even without addition of the 24:0 precursor as determined by Multivariate ANOVA with post-hoc Scheffe test (p<0.01, n=3). Similar results were obtained from ARPE-19 cells transduced with Ad5-mELOVL4 and supplemented with or without 24:0 fatty acid (FIGS. 3a&b). Expression of ELOVL4 in the ARPE-19 cells resulted in ^{˜}20- and 50-fold increases in 28:0 and 30:0 fatty acid, respectively, compared to controls.

These results have shown by the “gain of function” approach that, indeed, ELOVL4 protein is involved in elongation of saturated VLCFAs that are normally incorporated into sphingolipids and ceramides that are necessary for skin barrier permeability. This supports earlier findings in which ELOVL4 knockout results in neonatal lethality due to defects in skin barrier permeability in ELOVL4 knockout mice, while the heterozygote animals show reduction in levels of very long chain saturated fatty acids, ceramides and sphingolipids¹²⁻¹⁴. From these findings it was concluded that in the skin, ELOVL4 is responsible for synthesis of very long chain saturated fatty acids that are incorporated into ceramides and sphingolipids to protect the skin from dehydration.

In the retina, however, very long chain saturated fatty acids, sphingolipids and ceramides form a very minor component of the lipid pool²⁰. Instead, n-3/n-6 VLC-PUFA (C28-C40) are much more abundant and have been shown to be associated with rhodopsin, the major phototransduction protein in the retina^{21,22}. They can be synthesized from long chain polyunsaturated precursors when injected into the vitreal fluid. However, the elongase(s) responsible for their biosynthesis has never been shown²³. Based on the present data provided herein, it is now asserted that the ELOVL4 protein is an enzyme or a co-enzyme that plays a crucial role in biosynthesis of VLC-PUFAs found in the retina, brain, testis, and sperm (i.e., tissues that express the ELOVL4).

To confirm that ELOVL4 protein actually plays a role in biosynthesis of VLC-PUFAs (>C26), the ability of ELOVL4-transduced cells to elongate the VLC-PUFA precursors eicosapentaenoic acid (EPA; 20:5n3) and docosapentaenoic acid (DPA; 22:5n3) was examined. Cardiomyocytes transduced with ELOVL4 or GFP were treated for 72 h with 30 or 40 ug/mL of 20:5n3 or 22:5n3 fatty acid precursors. The results showed that all treatment groups elongated the precursors to C24-C26 n-3 fatty acids without any appreciable differences in their peak areas (FIG. 4a). However, ELOVL4-transduced cells also showed distinct peak sizes of the elongation products 28:5n3, 30:5n3, 32:5n3, 34:5n3, 36:5n3 and 38:5n3 fatty acids from either 20:5n3 or 22:5n3 precursor (FIGS. 4b&c). The control cells did not demonstrate these elongation steps (FIG. 4a). In the ELOVL4-transduced cells, the major VLC-PUFA products were 34:5n3 and 36:5n3 fatty acids. In addition, there were some 34:6n3 and 36:6n3 fatty acid products which are probably products from desaturase activities. These findings support observations that Stargardt disease-3 mutation in the mouse Elovl4 gene causes a reduction in the levels of C32-C36 acyl phosphatidylcholines¹⁸. Thus, Elovl4 is shown herein to be the gene responsible for biosynthesis of very long chain polyunsaturated fatty acids (beyond C26) that are incorporated into ceramides and sphingolipids and very long chain polyunsaturated fatty acid-containing phosphatidylcholines found in the retina, brain and testis, an activity which had not been established^{13-15,17,24} prior to the present disclosure.

Previous studies have also shown that the ELO families of proteins are enzymes involved in fatty acid elongation^{11,14,15}. Other mammalian ELOVLs that share similar homologies and specific conserved motifs such as dilysine for the endoplasmic reticulum (ER) retention motif (KXKXX) (SEQ ID NO:19), and a conserved histidine-rich motif (HXXHH) (SEQ ID NO:20) believed to act as an iron-chelating ligand used for electron transfer for O₂-dependent redox reactions^{3,24}, are involved in fatty acid biosynthesis. The present results establish for the first time ELOVL4 as a component of an elongation system that elongates both saturated and polyunsaturated fatty acids.

The results provided herein for cells that do not normally express ELOVL4 mRNA or protein show that ELOVL4 is necessary for the elongation to VLC-FA and VLC-PUFA. The alternative explanations provided above for the results obtained in mutated animals do not apply here, since there is no reason to suspect that the absence of ELOVL4 expression in normal cells functions to suppress an important biological activity in these cells. Without wishing to be bound by theory, a more logical explanation is that the myocytes and ARPE-19 cells have no biological need for the products of ELOVL4, and that the elongation products observed in both cell types transduced with ELOVL4 were the result of the direct participation of the ELOVL4 protein acting at some step in their elongation. Again, without wishing to be bound by theory, given the known roles of other ELOVL proteins as condensing enzymes, it is asserted herein that ELOVL4 catalyzes the addition of malonyl-CoA to C26 saturated and PUFA to form C28 compounds, using existing dehydrases and reductases shown to be involved in synthesis of fatty acids of all chain lengths and degrees of unsaturation. It is further asserted that ELOVL4 catalyzes condensation reactions in the formation of longer chain saturated and PUFA to generate the C30-C40 fatty acids characterized in the GC/MS analyses.

Methods

Construction of Recombinant Adenoviruses carrying mouse Elovl4: Recombinant adenovirus carrying the mouse Elovl4 gene (Ad5-mELOVL4) was constructed following Clontech's Adeno X Expression systems 2 with creator technology protocols (Clontech, Palo Alto, Calif.) and supplementary methods online. Recombinants were selected, sequenced, confirmed, purified, and digested with Pac I and then transfected into human embryonic kidney 293 cells (HEK-293) to generate virus particles. The recombinant viruses were prepared as high-titer stocks through the propagation in 293 cells by double cesium chloride purification, dialyzed against a 10 mM Tris (pH 8.0) buffer that contained 80 mM NaCl, 2 mM MgCl₂ and 10% glycerol²⁵. Infectious adenovirus titer was determined in triplicate by plaque forming assay and expressed as plaque-forming units (pfu) per ml.

Cell Cultures with Ad5-Mouse-ELOVL4: Rat neonatal cardiomyocytes were isolated from 1- or 2-day-old rats using the National Cardiomyocyte Isolation System (Worthington, Lakewood, N.J.). The isolated cells (5×10⁶ cells) were plated in 10-cm tissue culture plates and cultured in DMEM/F-12 (1:1) medium containing 10% (v/v) FBS and 5% (v/v) horse serum supplemented with 1 mM sodium pyruvate, penicillin (100 U/ml) and streptomycin (100 U/ml), at 37° C. in a tissue culture incubator with constant supply of 5% CO₂ in 95% relative humidity. Cells were used for experiments after two to three days in culture. ARPE-19 cells (2×10⁶) were cultured under the same conditions as myocytes.

Adenovirus infections of myocytes and ARPE-19 cells were carried out as described^{17,26}. After 24 h incubation, the infection medium was replaced with normal [15% (vol/vol) serum] culture medium supplemented separately with 50 or 100 μg/ml of the sodium salt of lignoceric acid (24:0) or 30 or 40 ug/ml of 20:5n3 or 22:5n3, which had been conjugated to bovine serum albumin fraction V²⁷. After incubation in lipid precursors for 48 or 72 h, cells were washed in phosphate buffered saline (PBS) containing 50 μM fatty acid free bovine serum albumin fraction V, then twice more with only PBS, scraped and stored as pellets at −80° C. until used.

RNA Isolation and cDNA Synthesis: RNA was isolated and purified from all the collected rat tissues, cultured cardiomyocytes, and ARPE-19 cells using the PureLink™ Micro-to-Midi Total RNA Purification System from Invitrogen (Carlsbad, Calif.) by following the manufacturer's protocol.

Production of Affinity-Purified ELOVL4 Antibodies: A synthetic 12-amino acid peptide corresponding to amino acids 301-312 of the mouse ELOVL4 protein was conjugated to keyhole limper hemocyanin and injected subcutaneously into rabbits for polyclonal antibodies production after collection of preimmune serum. Affinity purified antibodies were collected using immobilized peptide antigen (Bethyl Laboratory Inc, Montgomery, Tex.).

Immunofluorescence Labeling of Myocytes and ARPE-19 cells: Rat neonatal cardiomyocytes were sub-cultured onto poly-L-lysine coated four-well chamber glass slides. Twenty-four hours later, cells were transduced with Ad5-GFP or Ad5-mELOVL4, grown at 37° C. for 24 h, washed twice with ice cold PBS, and fixed at −20° C. in methanol:acetone (1:1, v/v) for 10 min. Ad5-GFP transduced cells were mounted with Vectashield mounting media (Vector laboratories, Inc., Burlingame, Calif.) for microscopy. Ad5-mELOVL4 transduced cells were blocked and permeabilized for 1 h at room temperature in PBS containing 10% (v/v) normal goat serum and 0.1% (v/v) Triton X-100, and treated with anti-ELOVL4 antibody (1:200) overnight at 4° C. Immunofluorescent labeling was visualized using a Zeiss Axioplan 2 fluorescence microscope.

Lipid Extraction and Fatty Acid Derivatization: Total cellular lipids were extracted as described³⁰. To the purified lipid extracts, a mixture of pentadecanoic acid (15:0), heptadecanoic acid (17:0), heneicosanoic acid (21:0), pentacosanoic acid (25:0), and heptacosanoic acid (27:0) was added for use as internal standards. 1.0 mL of 16.6% concentrated HCl in methanol was added and the tubes were sealed under N₂ with Teflon lined caps and heated at 75° C. for 5 hours. The tubes were cooled on ice and fatty acid methyl esters were extracted three times with 2.0 mL hexane. The hexane layers were dried under N₂, dissolved in 50 μl of hexane, sonicated, spotted on thin layer chromatography (TLC) plates, and developed in hexane:ether (80:20). The plates were stained with dichlorofluorescein; fatty acid methyl ester bands were then scraped from the TLC plates and collected. 2.0 mL of absolute ethanol were added to the scraped bands and the mixture was capped under N₂ and sonicated for 10 minutes. Fatty acid methyl esters were extracted three times with 2.4 mL hexane after adding 2.0 mL of de-ionized water. The combined hexane layers were dried under N₂ and dissolved in 20 μl nonane for GC-MS analysis.

FAME Analysis by GC-MS: Fatty Acid Methyl Esters (FAMES) were identified and quantified using an Agilent Technologies 6890N gas chromatograph (GC) with a 5975B inert XL mass spectrometer (MS) detector (Agilent Technologies, Wilmington, Del.). The GC-MS was operated in the electron impact (EI) single ion monitoring (SIM) mode. For saturated FAME analysis, the injection volume was 1 μl and the inlet, held at 320° C., was set to pulsed splitless mode. An Agilent Technologies HP-5 ms column (30 m×0.25 mm×0.25 u) was used with a helium carrier gas flow rate of 1.2 ml/min. The oven temperature began at 160° C., was ramped to 320° C. at 3° C./min, and held at 320° C. for 20 minutes. The MS transfer line, ion source and quadrupole temperatures were 320° C., 230° C., and 150° C., respectively. The 26:0, 28:0, and 30:0 response values were obtained using the m/z ratios 410.4, 438.4, and 466.5, respectively. Sample concentrations were determined by comparison to external standards, using 25:0 and 27:0 as internal standards. Multivariant ANOVA with post-hoc Scheffe tests determined statistical significance (p<0.05).

For very long polyunsaturated FAME analysis, the injection volume was 1 μl and the inlet, held at 290° C., was set to pulsed splitless mode. An SGE BPX70 column (35 m×0.22 mm×0.25 u) was used with a helium carrier gas flow rate of 1.2 ml/min (SGE, Austin, Tex.). The oven temperature began at 90° C. for 10 minutes, was ramped to 290° C. at 3° C./min, and held at 290° C. for 12.3 minutes. The MS transfer line, ion source and quadrupole temperatures were 290° C., 230° C., and 150° C., respectively. The PUFA response values were obtained using the m/z ratios 79.1, 108.1, and 150.1 in SIM mode. Abundances were compared by normalizing peak areas to nanomoles of 20:1, using 23:0 as an internal standard.

As used herein, “effective amount” means an amount of a VLC-PUFA component of the composition that is nontoxic but sufficient to provide the desired effect and performance at a reasonable benefit/risk ratio attending any dietary supplement or therapeutic composition.

Where used herein, the singular forms “a,” “an,” and “the” are understood to refer to the plural forms of the subject, unless the context clearly dictates otherwise.

The compositions and corresponding methods of the presently disclosed and claimed inventive concept(s) can comprise, consist of, or consist essentially of, the elements and limitations of the presently disclosed and claimed inventive concept(s) described herein, as well as any additional or optional ingredients, components, or limitations described herein or otherwise useful in compositions having nutritional and therapeutic applications as described herein.

### ABBREVIATIONS

Ad5, Adenovirus type 5; Ad5-mELOVL4, Ad carrying mouse Elovl4 gene; GFP, green fluorescent protein; Ad5-GFP, Ad5 carrying GFP gene; VLCFA, very long chain saturated fatty acid; VLC-PUFA, very long chain polyunsaturated fatty acid; ELOVL4, Elongation of Very Long Chain Fatty Acids-4; 24:0, lignoceric acid (tetracosanoic acid); 26:0, hexacosanoic acid; 28:0, octacosanoic acid/montanic acid; 30:0, triacontanoic acid/mellisic acid.

Although the presently disclosed and claimed inventive concept(s) has been described in detail, it should be understood that various changes, substitutions and alterations can be made herein without departing from the spirit and scope of the presently disclosed and claimed inventive concept(s) as defined by the appended claims. Moreover, the scope of the presently disclosed and claimed inventive concept(s) is not intended to be limited to the particular embodiments of the process, items of manufacture, compositions of matter, means, methods and steps described in the specification. As one of ordinary skill in the art will readily appreciate from the present disclosure, embodiments of the processes, items of manufacture, compositions of matter, means, methods, or steps, presently existing or later to be developed that perform substantially the same function or achieve substantially the same result as the corresponding embodiments described herein may be utilized according to the presently disclosed and claimed inventive concept(s). Accordingly, the appended claims are intended to include within their scope such processes, items of manufacture, compositions of matter, means, methods, or steps.

### REFERENCES

The following references, to the extent that they provide exemplary procedural or other details supplementary to those set forth herein, are specifically incorporated herein by reference.
1 K. Zhang, M. Kniazeva, M. Han et al., Nature genetics 27 (1), 89 (2001).
2 A. Maugeri, F. Meire, C. B. Hoyng et al., Investigative ophthalmology & visual science 45 (12), 4263 (2004); A. O. Edwards, L. A. Donoso, and R. Ritter, 3rd, Investigative ophthalmology & visual science 42 (11), 2652 (2001); T. R. Vrabec, A. Tantri, A. Edwards et al., American journal of ophthalmology 136 (3), 542 (2003).
3 C. Grayson and R. S. Molday, The Journal of biological chemistry 280 (37), 32521 (2005).
4 R. Ambasudhan, X. Wang, M. M. Jablonski et al., Genomics 83 (4), 615 (2004).
5 V. Vasireddy, C. Vijayasarathy, J. Huang et al., Molecular vision 11, 665 (2005).
6 S. Umeda, R. Ayyagari, M. T. Suzuki et al., Experimental animals/Japanese Association for Laboratory Animal Science 52 (2), 129 (2003).
7 P. Tvrdik, R. Westerberg, S. Silve et al., The Journal of cell biology 149 (3), 707 (2000).
8 C. S. Oh, D. A. Toke, S. Mandala et al., The Journal of biological chemistry 272 (28), 17376 (1997).
9 A. Jakobsson, R. Westerberg, and A. Jacobsson, Progress in lipid research 45 (3), 237 (2006).
10 R. Westerberg, J. E. Mansson, V. Golozoubova et al., The Journal of biological chemistry 281 (8), 4958 (2006); R. Westerberg, P. Tvrdik, A. B. Unden et al., The Journal of biological chemistry 279 (7), 5621 (2004).
11 A. E. Leonard, S. L. Pereira, H. Sprecher et al., Progress in lipid research 43 (1), 36 (2004); Y. Ge, Z. Chen, Z. B. Kang et al., Anticancer research 22 (2A), 537 (2002).
12 W. Li, R. Sandhoff, M. Kono et al., International journal of biological sciences 3 (2), 120 (2007).
13 A. McMahon, I. A. Butovich, N. L. Mata et al., Molecular vision 13, 258 (2007).
14 V. Vasireddy, Y. Uchida, N. Salem, Jr. et al., Human molecular genetics 16 (5), 471 (2007).
15 D. J. Cameron, Z. Tong, Z. Yang et al., International journal of biological sciences 3 (2), 111 (2007).
16 G. Karan, C. Lillo, Z. Yang et al., Proceedings of the National Academy of Sciences of the United States of America 102 (11), 4164 (2005).
17 W. Li, Y. Chen, D. J. Cameron et al., Vision research 47 (5), 714 (2007).
18 A. McMahon, S, N. Jackson, A. S. Woods et al., FEBS letters (2007).
19 M. N. Mandal, R. Ambasudhan, P. W. Wong et al., Genomics 83 (4), 626 (2004).
20 S. J. Fliesler and R. E. Anderson, Progress in lipid research 22 (2), 79 (1983).
21 M. I. Aveldano, Biochemistry 27 (4), 1229 (1988).
22 M. I. Aveldano and H. Sprecher, The Journal of biological chemistry 262 (3), 1180 (1987).
23 M. Suh and M. T. Clandinin, Current eye research 30 (11), 959 (2005).
24 T. Chertemps, L. Duportets, C. Labeur et al., Proceedings of the National Academy of Sciences of the United States of America 104 (11), 4273 (2007).
25 V. Denic and J. S. Weissman, Cell 130 (4), 663 (2007).
26 J. Xiao and J. Chodosh, Investigative ophthalmology & visual science 46 (10), 3777 (2005).
27 Z. B. Kang, Y. Ge, Z. Chen et al., Proceedings of the National Academy of Sciences of the United States of America 98 (7), 4050 (2001); Y. Ge, X. Wang, Z. Chen et al., Journal of neurochemistry 82 (6), 1360 (2002).
28 J. M. Street, D. W. Johnson, H. Singh et al., The Biochemical journal 260 (3), 647 (1989).
29 D. Raz-Prag, R. Ayyagari, R. N. Fariss et al., Investigative ophthalmology & visual science 47 (8), 3603 (2006); V. Vasireddy, M. M. Jablonski, M. N. Mandal et al., Investigative ophthalmology & visual science 47 (10), 4558 (2006).
30 R. E. Martin, M. H. Elliott, R. S. Brush et al., Investigative ophthalmology & visual science 46 (4), 1147 (2005).
31 Stoffel, W., Holz, B., Jenke, B., Binczek, E., Gunter, R. H., Kiss, C., Karakesisoglou, I., Thevis, M., Weber, A-A., Arnhold, S., and Addicks, K. Delta-6-Desaturase (FADS2) deficiency unveils the role of w3 and w6-polyunsaturated fatty acids. The EMBO Journal 2008 (27):2281-2292.
32 Agostoni, C. Role of long-chain polyunsaturated fatty acids in the first year of life. J Pediatr Gastroenterol Nutr. 2008 November; 47 Suppl 2:S41-4.

What is claimed is:

1. A method of enhancing fertility in a male or female subject in need of such therapy, the method comprising the step of:
administering to the male or female subject an effective amount of a composition comprising at least one purified very long chain polyunsaturated fatty acid (VLC-PUFA) having a chain length in a range of from 28 carbons to 40 carbons.

2. The method of claim 1, wherein the subject is male and wherein the composition is administered to the male subject as a nutritional supplement for enhancing at least one of sperm function and sperm viability of the male subject.

3. A method of enhancing at least one of sperm function and sperm viability of a male subject, the method comprising the step of:
adding a quantity of a composition comprising at least one purified very long chain polyunsaturated fatty acid (VLC-PUFA) having a chain length in a range of from 28 carbons to 40 carbons to a seminal fluid or sperm of the male subject.

4. A method of treating an ocular disorder in a subject in need of such therapy, the method comprising the step of:
administering to the subject an effective amount of a composition comprising at least one purified very long chain polyunsaturated fatty acid (VLC-PUFA) having a chain length in a range of from 28 carbons to 40 carbons.
